(19) United States
(12) Patent Application Publication
(10) Pub. No.: US 2008/0305559 A1
(43) Pub. Date: Dec. 11, 2008

(54) Non-Competitive Immunoassays to Detect Small Molecules

(76) Inventors: Gualberto Gonzalez-Sapienza, Montevideo (UY); Bruce Hammock, Davis, CA (US); Andres Gonzalez-Techera, Montevideo (UY); Lucia Vanrell, Montevideo (UY); Hee Joo Kim, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/097,235

(22) PCT Filed: Dec. 15, 2006
(86) PCT No.: PCT/US2006/048123

§ 371 Date: Jun. 12, 2008

Related U.S. Application Data
(60) Provisional application No. 60/750,921, filed on Dec. 15, 2005.

(51) Int. Cl. G01N 33/548

(52) U.S. Cl. 436/530

(57) ABSTRACT

The present invention provides noncompetitive immunoassays to detect small molecules.

### CROSS-REFERENCES TO RELATED APPLICATIONS

This application claims the benefit of U.S. Provisional Application No. 60/750,921, filed Dec. 15, 2005, the disclosure of which is incorporated by reference in its entirety for all purposes.

### STATEMENT AS TO RIGHTS TO INVENTIONS MADE UNDER FEDERALLY SPONSORED RESEARCH OR DEVELOPMENT

This invention was made with Government support under Grant No. TW05718, awarded by the NIH. The Government has certain rights in this invention.

### BACKGROUND OF THE INVENTION

Due to their simplicity, speed, low cost and specificity, immunoassays have become useful tools for the analysis of a variety of biological substances and small compounds such as environmental pollutants. The vast majority of immunoassays for small analytes such as pesticides, industrial organic pollutants, microbial toxins, abused drugs, hormones, and pharmaceuticals etc., have a competitive format, i.e., once the anti-hapten antibodies are produced, the same hapten or a structurally related molecule is conjugated to a tracer enzyme for the competition with analyte for the binding sites of immobilized antibody or coating protein to capture free antibody in the competition with analyte. Competitive-format assays are inferior to noncompetitive formats for which immobilized antibody on the solid support captures target molecule and another antibody conjugated with a signaling molecule detects the captured molecule in terms of sensitivity, precision, kinetics and working range, and are more difficult to adapt to rapid “on site” or “clinic” assays, such as dipsticks or immunochromatography.

Traditional methods for developing immunoassays start with hapten synthesis and production of anti-analyte antibodies by immunization of animals with a hapten-protein conjugate. The hapten is typically an analyte related compound modified to be covalently conjugated to the carrier protein. Once an analyte-specific, high titer anti-hapten serum is obtained, a competitive immunoassay (e.g., an ELISA) can be developed by using the same hapten coupled to a unrelated carrier protein. When the same hapten is used for immunization and coating, the assay is designated as homologous ELISA. However, very frequently, homologous assays are less sensitive than heterologous assays (the hapten used for immunization and coating are different). The design of heterologous assays requires extensive chemical synthesis work in order to develop a proper panel of candidate haptens, which must afterwards be tested, to examine whether the desired sensitivity can be reached. For the detection of small molecules, a sandwich type noncompetitive ELISA format is not applicable because once antibody binds to the target molecule, there is no site available for the direct binding of secondary reporter antibody. Nevertheless, there have been efforts into the development of noncompetitive ELISA for small molecules and limited successes have been reported using anti-immunocomplex antibodies or recombinant antibody techniques. However, these methods require considerable time consuming and laborious procedures such as production of primary antibody against target molecule, reimmunization of analyte-antibody complex to obtain anti immunocomplex antibody, and screening of a panel of antibodies or generation of recombinant antibody library and successive screening to select the one with affinity for the analyte-antibody complex.

Thus, there is a need in the art for new compositions and methods for detecting analytes, including small molecules. The present invention satisfies these and other needs.

### BRIEF DESCRIPTION OF THE DRAWINGS

FIG. 1 illustrates the noncompetitive immunoassays of the invention. An antibody-small molecule complex is formed by contacting a sample suspected of containing the small molecule with an antibody that specifically binds to the molecule. Once formed, the complex is further contacted with an affinity agent comprising a phage protein fused to a heterologous polypeptide that specifically binds to the antibody-small molecule complex, thereby forming an antibody-small molecule-affinity agent complex that can be detected due to the presence of the small molecule in the sample.

FIG. 2 illustrates data from phage titration curves for the development of a noncompetitive immunoassay to detect molinate. Phage suspensions of clones 8M2 (A) and 10M2 (B) were titrated on plates coated with 250 ng (circles), 130 ng (triangles), 60 ng (diamonds) or 30 ng (squares) per well of MoAb 14D7, in the presence of 100 ng/ml (black) or 0 ng/ml (white) of molinate.

FIG. 3 illustrates data demonstrating the effect of phage particle concentration on the noncompetitive ELISA for molinate. Different 8M2 phage concentrations were used to detect binding of molinate on plates coated with 50 ng/well of MoAb 14D7. The relative dilutions of the phage suspension are: 1:1000 (triangles), 1:500 (circles), 1:250 (starts), 1:125 (squares), 1:63 (diamonds), 1:20 (white circles).

FIG. 4 illustrates data from noncompetitive molinate ELISA set up with different phage borne peptides. Phage clones bearing each of the four sequences isolated in the panning experiments were used. The SC₅₀ values were: clone 1M (circles)=5.0±0.4 ng/ml, clone 8M2 (triangles)=6.1±0.3 ng/ml, clone 2EM (squares)=32∓3 ng/ml and clone 10M2 (diamonds)=32±5 ng/ml.

FIG. 5 is a table which depicts data confirming the specificity of the noncompetitive assays described herein.

FIG. 6 illustrates data from a phage dipstick assay for molinate. Decreasing amounts of MoAb 14D7 were immobilized on nitrocellulose strips and dipped into 0, 20 and 100 ng/ml of molinate in water. From top to bottom: 8, 4, 2 and 1 ng of MoAb 14D7 per spot.

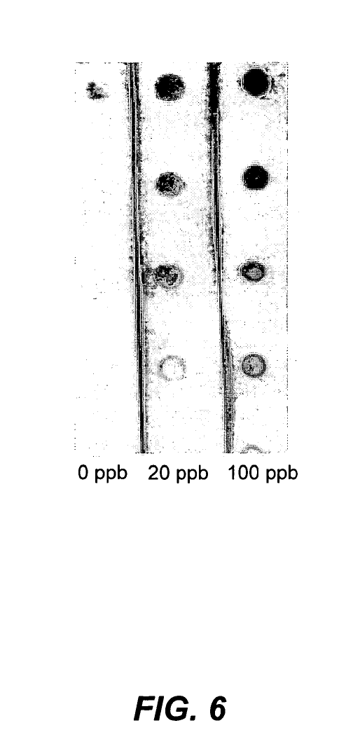

FIG. 7 illustrates data from a noncompetitive assay to detect atrazine. mAb K4E7 was immobilized on ELISA plates and three phage borne peptides (clones 4A, squares; 14A, circles; 12A, triangles) were used as tracers. Phage particles were detected with anti-M13 HRP conjugate.

FIG. 8 depicts the pAFF/mBAP vector showing the cloning oligonucleotides in frame with pIII gene. Top: pAFF/mBAP vector map: araC, arabinose represor gene; pIII, pIII phage coat protein gene; mBAP, mutated form of BAP gene with increased kcat; bla, β-lactamase gene. Middle: The oligonucleotide encoding the random peptides are cloned between the two BstxI restriction sites. Bottom: Annealing of purified linear BstxI digested pAFF/mBAP vector with the library coding oligonucleotide and the ON-28 and ON-29 adaptor oligonucleotides. The aminoacid sequence encoded by the assembled construct is shown at the bottom.

FIG. 9 illustrates data demonstrating the reactivity of phage clones with the immunocomplex and the anti-PBA affinity purified antibody. Phage supernatants were added to wells coated with BSA (black), affinity purified anti-PBA antibodies (strips), or affinity purified anti-PBA antibodies in the presence of 100 ng/ml of PBA. Supernatants isolated in the last round of panning (1-10); helper phage supernatant (11).

FIG. 10 illustrates data demonstrating the reactivity of the phage borne peptide with the PBA-antibody immunocomplex using different amounts of coating antibody. Plates were coated with 10 μg/ml protein G purified antibody, 10a; 5 μg/ml, 10b; 2.5 μg/ml, 10c; or 1.25 μg/ml, 10d; and incubated with serial dilutions of phage borne peptide in the presence (squares) or absence (circles) of 50 ng/ml of PBA.

FIG. 11 illustrates data from a noncompetitive ELISA for PBA performed in plates coated with 10 μg/ml protein G purified IgG yielding a SC₅₀=0.31±0.03 ng/ml (SC₅₀=50% of maximal signal).

FIG. 12 illustrates the reactivity of the phage borne peptides with the IC and the uncombined antibody. Serial dilutions of clones 1M (left) or mA (right) were assay on ELISA plates coated with 500 ng (diamonds), 250 ng (squares), 125 ng (triangles), or 63 ng per well (circles) of MoAb 14D7, in the presence of 50 ng/ml of molinate (black) or in its absence.

FIG. 13 illustrates a dipstick assay for molinate using phage borne peptides. Top panel: different amounts of MoAb 14D7 (ranging from 2-0.03 μg) spotted onto nitrocellulose strips were incubated with the 10¹⁰ phage particles bearing peptides 1M or mA in the presence (+) or absence (−) of 50 ng/ml of molinate. The bottom panel shows the results of the dispstick assay set up with 30, 15 or 7.5 ng of MoAb 14D7 and clone 1M, in the range of 0 to 80 ng/ml of molinate.

FIG. 14 illustrates PHAIA for atrazine using different phage borne peptides. Noncompetitive assay set up with MoAb 4KE7 and different phage borne peptides: 4A (dark circles), 9A (diamonds), 11A (white squares), 12A (black squares), 13A (white circles) and 14A (triangles). The sequences and SC₅₀ values for the different peptides are shown in the insert. Standard curves were normalized by expressing experimental absorbance values (A) as (A/A_{M}), where A_{M} is the maximum absorbance value of the assay.

FIG. 15 illustrates the reactivity of the phage borne peptide with the PBA IC using different amounts of coating antibody. Plates were coated with 10 μg/ml of protein G-purified antibodies, 2a; 5 μg/ml, 2b; 2.5 μg/ml, 2c; or 1.25 μg/ml, 2d; and incubated with serial dilutions of phage borne peptide in the presence (black circles) or absence (white circles) of 50 ng/ml of PBA.

FIG. 16 illustrates dipstick PHAIA for PBA. Left panel: protein G-purified anti-PBA antibodies (ranging from 3 to 0.09 μg) spotted onto nitrocellulose strips were incubated with different concentrations of phage particles/ml (as indicated on the vertical axis) in the presence (+) or absence (−) of 50 ng/ml of PBA. Right panel: Dipstick assay set up with various amounts of protein G-purified anti-PBA antibodies (3 to 0.19 μg/spot) and using 5×10¹¹ phage particles/ml for detection. The concentration of PBA ranged from 8 to 0 ng/ml as shown in the top of the right panel.

### SUMMARY OF THE INVENTION

The present invention provides a method for converting noncompetitive immunoassays to competitive immunoassays thus increasing sensitivity and speed of the assay and making it easier to adapt to many commonly used formats for improving the sensitivity of competitive detection methods for analytes.

One embodiment of the invention provides a method for detecting an analyte by (a) contacting a sample suspected of containing the analyte with a first antibody that specifically binds to the analyte, thereby forming an antibody-analyte complex; (b) contacting the antibody-analyte complex with an affinity agent comprising a phage protein fused to a heterologous polypeptide, wherein the heterologous polypeptide specifically binds to the antibody-analyte complex, thereby forming an antibody-analyte-affinity agent complex; and (c) detecting the antibody-analyte-affinity agent complex, thereby detecting the analyte. The first antibody may be a monoclonal antibody or a polyclonal antibody or an antibody fragment (e.g., Fab). The technology also applies to ligand receptor based assays in general. Phage-borne polypeptide specific to receptor-ligand or receptor-protein complexes can be used to quantitatively detect bound ligand or protein. The heterologous polypeptide portion of the affinity agent may bind to the antibody or receptor at an allosteric site, adjacent to the molecule binding sites, or at sites bridging the antibody-small molecule or receptor-ligand/protein sites. The first affinity agent may comprise phage coat protein pVIII or pIII. The antibody-analyte-affinity agent or receptor-ligand/protein complex may be detected by contacting the complex of step (b) with a second antibody that specifically binds to a phage protein. The second antibody may further comprise a detectable label (e.g., a fluorescent label, a radiolabel, or an enzymatic label). The analyte may be molinate, atrazine, or phenoxybenzoic acid. In some embodiments, the analyte is molinate and the heterologous polypeptide comprises one of the following sequences: WDT or X1X2X3WDTX4×5, wherein X1 is selected from the group consisting of: C and R; X2 is selected from the group consisting of: S and N; X3 is selected from the group consisting of: T, R, H, and V; X4 is selected from the group consisting of: T, W, and S; and X5 is selected from the group consisting of: G and C. In some embodiments the analyte is atrazine and the heterologous polypeptide comprises one of the following sequences: WFD or X1WFDX2X3, wherein X1 is selected from the group consisting of: R, S, Y, and M; X2 is selected from the group consisting of: N, E, A, L, and M; and X3 is selected from the group consisting of: S, G, P, C, and Y. In some embodiments, the analyte is phenoxybenzoic acid and the heterologous polypeptide comprises the following sequence: CFNGKDWLYC. In various embodiments, the analyte maybe a small molecule or a larger molecule, such as oligosaccharides, proteins, ligands or receptors, an epitope for an antibody, or any molecular entity that undergoes binding.

Another embodiment of the invention provides kits for detecting analytes. In some aspects, kits can comprise a first antibody immobilized to a solid support, such as a membrane or multiwell plate. The membrane may be nitrocellulose. In some embodiments, the solid support may be in the form of a dipstick.

### DETAILED DESCRIPTION OF THE INVENTION

### I. Introduction

Due to their excellent sensitivity and specificity, immunodetection techniques, which use the fine specificity of antibodies to detect trace amounts of target compounds, are particularly suitable for a wide range of applications in biomedical and environmental analysis. Depending on the format, the assays fall into two main categories, noncompetitive and competitive immunoassays. Noncompetitive assays, mostly used for large molecules and proteins, are based on two different antibodies able to bind simultaneously to two independent epitopes on the analyte (two-site assays). Analysis of low-molecular-mass analytes such as drugs, hormones, toxins, pesticides, explosives, etc., not large enough to bind two antibodies simultaneously, requires a competitive immunoassay format. In this format, the analyte competes with a labelled (or immobilized) analyte analogue (hapten), and the measured signal is inversely proportional to the concentration of analyte in the sample. In this way, the formation of the analyte-antibody immunocomplex (IC) is indirectly quantitated by measuring the empty binding sites of the unreacted antibody. Thus, in order to maximize the change in signal when trace amounts of analyte are analyzed, the amount of antibody has to be minimized; this in turn, poses a limitation to the assay sensitivity. This is supported by mathematical modeling of immunoassay performance, which also shows that competitive assays are inferior to noncompetitive ones in terms of precision, kinetics, and working range (see, e.g., Jackson, T. M. and R. P. Ekins, Theoretical limitations on immunoassay sensitivity. Current practice and potential advantages of fluorescent Eu3+ chelates as non-radioisotopic tracers. J. Immunol. Methods, 1986, 87(1): p. 13-20). In addition, noncompetitive immunoassays are more easily adapted into rapid ‘on-site’ formats like dipstick or immunochromatography, as well as to microfluidics and biosensors. For this reason, different attempts have been made to implement small-molecule noncompetitive assays, but they have usually been limited to certain chemical structures or require analyte labeling (see, e.g., Pradelles, P., et al., Immunometric assay of low molecular weight haptens containing primary amino groups. Anal. Chem., 1994, 66(1): p. 16-22; Piran, U., W. J. Riordan, and L. A. Livshin, New noncompetitive immunoassays of small analytes. Clin Chem, 1995, 41(7): p. 986-90).

A method based on the blockage of the unreacted sites of the antibody with a polydentate ligand has been described (see, e.g., Giraudi, G., et al., A general method to perform a noncompetitive immunoassay for small molecules. Anal. Chem., 1999, 71(20): p. 4697-700), and successfully applied to the detection of cortisol, but a major drawback appears to be the difficulty in obtaining a stable blockage of the unoccupied antibody binding sites. An alternative is the development of the so called open-sandwich enzyme-linked immunosorbent assay (ELISA), an immunoassay based on antigen-dependent stabilization of antibody variable regions (V(H) and V(L) domains). In this format, the analyte-dependent association of the antibody domains is used to bring together a tracer enzymatic activity, such as by joining the N- and C-terminal domains of beta-galactosidase (see, e.g., Yokozeki, T., et al., A homogeneous noncompetitive immunoassay for the detection of small haptens. Anal. Chem., 2002, 74(11): p. 2500-4). The open-sandwich ELISA is quick and highly sensitive, but the technology is laborious and requires a stringent control of the background association of the recombinant V(H) and V(L) domains in the absence of analyte, which is again case specific.

The most common approach for the development of noncompetitive ELISAs for small-molecules relies on the use of anti-immune complex antibodies (see, e.g., Ullman, E. F., et al., Anti-immune complex antibodies enhance affinity and specificity of primary antibodies. Proc. Natl. Acad. Sci. USA, 1993, 90(4): p. 1184-9; Self, C. H., J. L. Dessi, and L. A. Winger, High-performance assays of small molecules: enhanced sensitivity, rapidity, and convenience demonstrated with a noncompetitive immunometric anti-immune complex assay system for digoxin. Clin. Chem., 1994, 40(11 Pt 1): p. 2035-41), however these antibodies are difficult to obtain. In general, when antibodies are elicited against the antigenic determinant of an antibody's combining site (idiotope), the interface of the idiotope-antiiditope antibody complex buries a large surface that involves most of the complementarity determining regions (CDRs) of both antibodies (see, e.g., Pan, Y., S. C. Yuhasz, and L. M. Amzel, Anti-idiotypic antibodies: biological function and structural studies. FASEB J., 1995, 9(1): p. 43-9). When the idiotope under consideration corresponds to that of an anti-hapten antibody, it is important to consider that its modification upon reaction with the hapten will be modest and restricted to the binding pocket. In these antibodies, up to 85% of the accessible surface of the hapten can be buried after binding (see, e.g., Lamminmaki, U. and J. A. Kankare, Crystal structure of a recombinant anti-estradiol Fab fragment in complex with 17beta-estradiol. J. Biol. Chem., 2001, 276(39): p. 36687-94; Monnet, C., et al., Highly specific anti-estradiol antibodies: structural characterisation and binding diversity. J. Mol. Biol., 2002, 315(4): p. 699-712), and therefore the small portion of the hapten that remains exposed to the solvent has little contribution to the formation of the new idiotope. This imposes a serious limitation to the preparation of anti-immune complex antibodies for two-site assay development, because the residual affinity of the anti-immune complex antibody for the free site of the anti-hapten antibody can be significant, and a major cause of high background noise in the assay. This is probably the main impediment for the widespread application of anti-immune complex antibodies for small-analyte immunodetection, which explains why after the initial applications described by Ullman and Self, only a limited number of additional applications have been reported (see, e.g., Towbin, H., et al., Sandwich immunoassay for the hapten angiotensin II. A novel assay principle based on antibodies against immune complexes. J Immunol Methods, 1995, 181(2): p. 167-76; Nagata, S., et al., A new type sandwich immunoassay for microcystin: production of monoclonal antibodies specific to the immune complex formed by microcystin and an anti-microcystin monoclonal antibody. Nat Toxins, 1999, 7(2): p. 49-55; Kobayashi, N., et al., Monoclonal anti-idiotype antibodies recognizing the variable region of a high-affinity antibody against 11-deoxycortisol. Production, characterization and application to a sensitive noncompetitive immunoassay. J Immunol Methods, 2003, 274(1-2): p. 63-75; Kobayashi, N., et al., Idiotype-anti-idiotype-based noncompetitive enzyme-linked immunosorbent assay of ursodeoxycholic acid 7-N-acetylglucosaminides in human urine with subfemtomole range sensitivity. J Immunol Methods, 2003, 272(1-2): p. 1-10).

In order to overcome this limitation, we sought “to focus” the recognition of the IC to the region of the idiotope where major changes are produced after binding of the hapten. For this, we substituted the large surface of the antiidiotope antibody binding site by short peptide loops that specifically react with the exposed region of the hapten and the conformational changes caused by its binding. For the selection of these peptides, we used phage display peptide libraries, reviewed by Smith and Petrenko (see, e.g., Smith, G. P. and V. A. Petrenko, Phage Display. Chem. Rev, 1997, 97(2): p. 391-410), which have been shown to be an excellent source of peptide ligands for a large number of selector molecules. We demonstrated the potential of this approach using the herbicides molinate and atrazine, as well as, insecticide metabolite, phenoxybenzoic acid (PBA), as model analytes, and a phagemid library expressing 7 and 8-mer cyclic random peptides. Due to the control that can be exerted during the high throughput selection process, the isolation of these peptides is more systematic and much less laborious that the trial-and-error process of anti-immunocomplex antibody preparation.

The present invention provides a noncompetitive immunoassay for detecting small molecules. Antibodies that specifically bind to small molecules were generated and peptides that specifically bind to antibody-small molecule complexes were selected from complex phage display peptide libraries. Each of these components were used in the noncompetitive immunoassays described herein to detect the small molecules.

We have explored the use of analyte peptidomimetics selected from phage display libraries as convenient substitutes of competing haptens. Phage display libraries provide a tool by which billions of different peptides can be expressed in the surface of filamentous phage. This permits billions of different peptides to be screened, simultaneously, for binding to the target of interest. Once an animal is immunized and a specific high titer serum is achieved, affinity purification of specific the anti-analyte antibodies using the same hapten used for immunization, followed by panning in the presence of analyte as presented in this work, provides a straightforward and systematic strategy to attain highly sensitive assays, in which the signal is proportional to the concentration of analyte. In the present invention, we have selected short peptide loops that specifically bind to the analyte-antibody complex and can thus be used to detect the analyte in a noncompetitive assay format. Without being bound by any particular theory, the binding of the peptides to the analyte-antibody complex increases the affinity of the analyte-antibody complex. Since noncompetitive assays are superior to competitive ones in terms of sensitivity, precision and kinetics, the methods described herein are an attractive novel approach for the development of noncompetitive immunoassays to detect small analytes.

### II. Definitions

“Analyte” or “small molecule” as used herein refers a compound to be detected. Small molecules and analytes include, e.g., pesticides, industrial organic pollutants, microbial toxins, drugs (e.g., illegal drugs such narcotics), hormones, explosives, dyes, and plasticizers. Industrial organics include polyhalogenated biphenyls, dioxins, dibenzofurans, aromatic ethers, ureas, aromatic amines, and may other pyrethroids. Pesticides include, e.g., thiocarbamates, phosphate esters, thiophosphates, carbamates, polyhalogenated sulfonamides, and their metabolites and derivatives. Drugs include, e.g., alkaloids such as morphine alkaloids such as, for example, morphine, codeine, heroin, dextromethorphan, cocaine alkaloids, such as, e.g., cocaine and benzoylecgonine; ergot alkaloids, which include the diethylamide of lysergic acid; steroid alkaloids; iminazoyl alkaloids; quinazoline alkaloids; isoquinoline alkaloids; quinoline alkaloids, which include quinine and quinidine; diterpene alkaloids; lactams including, e.g., barbiturates such as, phenobarbital and secobarbital, diphenylhydantoin, primidone, ethosuximide,; aminoalkylbenzenes, including, e.g., amphetamines; catecholamines such as ephedrine, L-dopa, epinephrine; narceine; papaverine; and their metabolites; benzheterocyclics, including, e.g., oxazepam, chlorpromazine, tegretol, their derivatives and metabolites; purines, including, e.g., which theophylline and caffeine; drugs derived from marijuana, including, e.g., cannabinol and tetrahydrocannabinol; polypeptides such as angiotensin, LHRH, and immunosuppresants such as cyclosporin, prograf, digosin, FK506, mycophenolic acid, and the like; vitamins such as A, B, e.g. B12, C, D, E and K, folic acid, thiamine; prostaglandins; tricyclic antidepressants, which include imipramine, dismethylimipramine, amitriptyline, nortriptyline, protriptyline, trimipramine, chlomipramine, doxepine, and desmethyldoxepin; anti-neoplastics, including, e.g., methotrexate; antibiotics, including, e.g., penicillin, chloromycetin, actinomycetin, tetracycline, terramycin. Hormones include, e.g., thyroxine, cortisol, triiodothyronine, testosterone, estradiol, estrone, progestrone, steroids, including, e.g., estrogens, androgens, andreocortical steroids, bile acids, cardiotonic glycosides and aglycones, which includes digoxin and digoxigenin, saponins and sapogenins, and steroid mimetic substances, such as diethylstilbestrol. Explosives include, e.g., 2, 4, 6 trinitrotoluene and a wide variety of alkyl and arynitro compounds.

It will be understood by the skilled artisan that an analyte can include a variety of small molecules, as well as larger molecules, including oligosacchrides, proteins, and in general, an epitope for an antibody. An analyte can include any molecular entities that undergo binding, such as receptors and ligands.

A “sample” as used herein refers to a sample of any source which is suspected of containing a small molecule. These samples can be tested by the methods described herein and include, e.g., water from an ocean, lake, river, pond, or stream, runoff water from an agricultural field, waste stream from an industrial operation (e.g., a mine, a heating plant, or a manufacturing plant) cosmetics, human food, nutraceuticals. A sample can be from a laboratory source or from a non-laboratory source. A sample may be suspended or dissolved in liquid materials such as buffers, extractants, solvents and the like. Samples also include animal and human body fluids such as whole blood, blood fractions, serum, plasma, cerebrospinal fluid, lymph fluids, milk; and biological fluids such as tissue and cell extracts, cell culture supernatants; fixed tissue specimens; and fixed cell specimens.

“Antibody” refers to a polypeptide encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Antibodies are representative of a wide variety of receptors including hormone receptors, drug targets such as peripheral benzodiazepine receptor, and carrier proteins.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab′)2, a dimer of Fab which itself is a light chain joined to VH-CH1 by a disulfide bond. The F(ab′)2 may be reduced under mild conditions to break the disulfide linkage in the hinge region, thereby converting the F(ab′)2 dimer into an Fab monomer. The Fab monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms, such as other mammals, may be used to express humanized antibodies. Alternatively, phage display technology can be used to identify antibodies, heteromeric Fab fragments and other proteins that specifically bind to selected antigens (see, e.g., McCafferty et al., Nature 348:552-554 (1990); Marks et al., Biotechnology 10:779-783 (1992)).

The phrase “specifically (or selectively) binds” when referring to an antibody refers to a binding reaction that is determinative of the presence of the antibody's binding target (e.g., a small molecule, protein epitope, or a phage protein) in a heterogeneous population of analytes (e.g., small molecules, phage proteins, and other biologics). Thus, under designated immunoassay conditions, the specified antibodies bind to their binding target at least two times the background and do not substantially bind in a significant amount to other analytes present in the sample. Specific binding to an antibody under such conditions may require an antibody that is selected for its specificity for a particular target (e.g., a small molecule, protein epitope, or a phage protein). For example, monoclonal and polyclonal antibodies raised to fusion proteins can be selected to obtain only those monoclonal and polyclonal antibodies that are specifically immunoreactive with a fusion protein and not with individual components of the fusion proteins. This selection may be achieved by subtracting out antibodies that cross-react with the individual antigens. A variety of immunoassay formats may be used to select antibodies specifically immunoreactive with a particular target (e.g., a small molecule or a phage protein). For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Affinity agent” as used herein refers to a phage protein fused to a heterologous polypeptide that specifically binds to an antibody-small molecule complex.

“Phage protein” as used herein refers to proteins from a filamentous phage and include, e.g., major coat protein (pVIII), particle assembly proteins (pVII and pIX), particle stability and infectivity proteins (pVI and pIII), and phage assembly and transport proteins (pI and pIV).

The term “heterologous” when used with reference to a protein indicates that the protein comprises two or more subsequences that are not found in the same relationship to each other in nature (e.g., a fusion protein between a phage protein and a polypeptide that specifically binds to an antibody-small molecule complex).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., any carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

A “label” or “detectable label” is a composition detectable by spectroscopic, photochemical, biochemical, immunochemical, or chemical means. For example, useful labels include radioisotopes (e.g., ³H, ³⁵S, ³²P, ⁵¹Cr, or ¹²⁵I), fluorescent dyes, ESA signals, electron-dense reagents, enzymes (e.g., alkaline phosphatase, horseradish peroxidase, or others commonly used in an ELISA), biotin, digoxigenin, or haptens and proteins for which antisera or monoclonal antibodies are available. For example, an antibody that specifically binds to a small molecule or a phage polypeptide can be made detectable, e.g., by incorporating a fluorescent into the antibody. The label can then be used to detect the presence of the small molecule or phage polypeptide in a sample.

### III. Methods of the Invention

In one aspect, the present invention provides a noncompetitive assay (e.g., an immunoassay) for detecting small molecules. Antibodies that specifically bind to a small molecule of interest (e.g., a pesticide, an industrial organic pollutants, a microbial toxin, a hormone, or a drug, including, e.g., illegal drugs such as narcotics) are contacted with a sample suspected of containing the small molecule. If present, the small molecule forms a complex with the antibody. The complex is then contacted with an affinity agent comprising a phage protein fused to a heterologous polypeptide that specifically binds to the small molecule-antibody complex. The complex is detected, thereby detecting the small molecule.

A. Antibodies that Specifically Bind Small Molecules

For preparation of antibodies, e.g., recombinant, monoclonal, or polyclonal antibodies, any technique known in the art can be used (see, e.g., Kohler & Milstein, Nature 256:495-497 (1975); Kozbor et al., Immunology Today 4: 72 (1983); Cole et al, pp. 77-96 in Monoclonal Antibodies and Cancer Therapy, Alan R. Liss, Inc. (1985); Coligan, Current Protocols in Immunology (1991); Harlow & Lane, supra; and Goding, Monoclonal Antibodies: Principles and Practice (2d ed. 1986)). Generation and screening of antigen specific polyclonal antibodies is described in, e.g., Harlow and Lane, supra. Generation of monoclonal antibodies has been previously described and can be accomplished by any means known in the art. (see, e.g., Buhring et al. in Hybridoma 1991, Vol. 10, No. 1, pp. 77-78). Generation of monoclonal antibodies against small molecules has been described in, e.g., Rufo et al., J. Ag. Food Chem. 52:182-187 (2004). For example, an animal such as a guinea pig or rat, preferably a mouse, is immunized with a small molecule conjugated to a hapten (e.g., KLH), the antibody-producing cells, preferably splenic lymphocytes, are collected and fused to a stable, immortalized cell line, preferably a myeloma cell line, to produce hybridoma cells which are then isolated and cloned. (see, e.g., U.S. Pat. No. 6,156,882). In addition, the genes encoding the heavy and light chains of a small molecule-specific antibody can be cloned from a cell, e.g., the genes encoding a monoclonal antibody can be cloned from a hybridoma and used to produce a recombinant monoclonal antibody. Specific monoclonal antibodies will usually bind with a K_{D} of at least about 10⁻⁸ M, more usually at least about 10⁻¹⁰ M; and most preferably, about 10⁻¹² M or better.

B. Peptides that Specifically Bind to Antibody-Small Molecule Complexes

Affinity agents of the invention comprise a phage protein and a polypeptide that specifically bind to an antibody-small molecule complex. The affinity agents described herein can be identified using phage display technology (see, e.g., Cardozo et al., Env. Sci. Tech. 39(100):4234 (2005); Goldman et al., Analytica Chimica Acta 457:13-19 (2002); McCafferty et al., Nature 348:552-554 (1990); Marks et al., Biotechnology 10:779-783 (1992)). A phage display system is a system in which polypeptides of interest are expressed as fusion proteins on the phage surface (Pharmacia, Milwaukee Wis.). Phage display can involve the presentation of a polypeptide sequence that specifically binds to a complex between an antibody and a small molecule on the surface of a filamentous bacteriophage, typically as a fusion with a bacteriophage coat protein (e.g., pVIII or pIII).

Generally in these methods, each phage particle or cell serves as an individual library member displaying a single species of displayed polypeptide in addition to the natural phage or cell protein sequences. The plurality of nucleic acids are cloned into the phage DNA at a site which results in the transcription of a fusion protein, a portion of which is encoded by the plurality of the nucleic acids. The phage containing a nucleic acid molecule undergoes replication and transcription in the cell. The leader sequence of the fusion protein directs the transport of the fusion protein to the tip of the phage particle. Thus, the fusion protein that is partially encoded by the nucleic acid is displayed on the phage particle for detection and selection by the methods described above and below. For example, the phage library can be incubated with a predetermined (desired) ligand (e.g., an antibody-small molecule complex), so that phage particles which present a fusion protein sequence that binds to the ligand can be differentially partitioned from those that do not present polypeptide sequences that bind to the predetermined ligand. For example, the separation can be provided by immobilizing the predetermined ligand. The phage particles (i.e., library members) which are bound to the immobilized ligand are then recovered and replicated to amplify the selected phage subpopulation for a subsequent round of affinity enrichment and phage replication. After several rounds of affinity enrichment and phage replication, the phage library members that are thus selected are isolated and the nucleotide sequence encoding the displayed polypeptide sequence is determined, thereby identifying the sequence(s) of polypeptides that bind to the predetermined ligand. Such methods are further described in WO 91/17271, 91/18980, and WO 91/19818 and WO 93/08278.

Examples of other display systems include ribosome displays, a nucleotide-linked display (see, e.g., U.S. Pat. Nos. 6,281,344; 6,194,550, 6,207,446, 6,214,553, and 6,258,558), polysome display, cell surface displays and the like. The cell surface displays include a variety of cells, e.g., E. coli, yeast and/or mammalian cells. When a cell is used as a display, the nucleic acids, e.g., obtained by PCR amplification followed by digestion, are introduced into the cell and translated.

Those of skill in the art will recognize that the steps of generating variation and screening for a desired property can be repeated (i.e., performed recursively) to optimize results. For example, in a phage display library or other like format, a first screening of a library can be performed at relatively lower stringency, thereby selected as many particles associated with a target molecule (e.g. a particular antibody-small molecule complex) as possible. The selected particles can then be isolated and the polynucleotides encoding the polypeptide that specifically binds to the target molecule can be isolated from the particles. Additional variations can then be generated from these sequences and subsequently screened at higher affinity. Specific heterologous polypeptides will usually bind with a K_{D} of at least about 10⁻⁸ M, more usually at least about 10⁻¹⁰ M; and most preferably, about 10⁻¹² M or better.

C. Detecting the Complex

The antibody-small molecule-affinity agent complex can be detected using any means known in the art. In some embodiments, the affinity agent is labeled and detection of the label detects the antibody-small molecule-affinity agent complex. In some embodiments, a secondary detection molecule (e.g., an antibody) that specifically binds to the phage protein is contacted with the antibody-small molecule-affinity agent complex. Detection of the secondary detection molecule detects the antibody-small molecule-affinity agent complex, thereby detecting the small molecule in the sample. In some embodiments, the secondary detection molecule is labeled with a detectable label.

The particular label or detectable group used in the assay is not a critical aspect of the invention, as long as it does not significantly interfere with the specific binding of the antibody to the small molecule or phage protein. The detectable group can be any material having a detectable physical or chemical property. Thus, a label is any composition detectable by spectroscopic, photochemical, biochemical, electrical, optical or chemical means. A wide variety of labels may be used, with the choice of label depending on sensitivity required, ease of conjugation with the compound, stability requirements, available instrumentation, and disposal provisions. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), and calorimetric labels such as colloidal gold or colored glass or plastic beads (e.g., polystyrene, polypropylene, latex, etc.).

The molecules can be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see, e.g., U.S. Pat. No. 4,391,904.

Means of detecting labels are well known to those of skill in the art. Thus, for example, where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. For example, protein compositions such as bovine serum albumin (BSA), nonfat powdered milk, and gelatin are widely used with powdered milk can be used.

### IV. Kits

The invention further provides kits for detecting small molecules. Such kits typically comprise two or more components necessary for measuring, and/or detecting small molecules. Components may be compounds, reagents, containers and/or equipment. For example, one container within a kit may contain an antibody that specifically binds to a small molecule of interest, and another container within a kit may contain an affinity reagent comprising a phage polypeptide fused to a heterologous polypeptide that specifically binds to a complex between an antibody and a small molecule. In addition, the kits comprise instructions for use, i.e., instructions for using the components in the noncompetitive immunoassays as described herein.

### EXAMPLES

The embodiments of the present invention are further illustrated by the following examples. These examples are offered to illustrate, but not to limit the claimed invention.

### Example 1

### Development of a Noncompetitive Immunoassay to Detect Molinate

Selection of Peptides that React with the Antibody-Analyte Complex

For the selection of peptides that react with the immunocomplex but do not react with the free antibody, we used a peptide library expressed in the coat protein pVIII, composed of hepta- and octa-peptides constrained by a disulfide bridge. During the selection (panning) process, we included adsorption steps with the uncombined antibody. One model system has been based on the use of the monoclonal antibody (MoAb) 14D7 specific for the herbicide molinate.

Briefly, the phage display peptide library is initially panned with the immunocomplex (immobilized antibody that has been previously incubated with an excess of the analyte). After binding and washing, the bound phage particles are eluted using acidic conditions, neutralized, and adsorbed on ELISA plate wells coated with antibody. The latter step is introduced to remove unwanted peptides that might react with the empty antigen binding site of the antibody. After amplification of the selected phage pool, the panning steps are repeated 3-4 times to enrich for the desired clones, and finally, individual phage clones are selected and assayed. Table 1 shows the sequences of phage clones isolated from different panning experiments on the MoAb 14D7-molinate complex. Only 4 different sequences were obtained out of 20 clones sequenced, and all have the consensus sequence WDT, shown in bold in the figure. While this motif seems to be required for binding to the antibody-analyte complex, the flanking residues appeared to be crucial for the performance of the peptides.

TABLE 1

Peptide sequences isolated with the MoAb
14D7-molinate complex.

| SEQUENCE | | |
|---|---|---|
| ID | phage clone | peptide sequence |
| S1 | 1M | C S T W D T T G W C |
| | 10M | C S T W D T T G W C |
| S2 | 4M2 | C N R W D T T G W C |
| | 5M2 | C N R W D T T G W C |
| | 8M2 | C N R W D T T G W C |
| | 11M2 | C N R W D T T G W C |
| | 13M2 | C N R W D T T G W C |
| S3 | 6M | C R S H W D T W C |
| | 8M | C R S H W D T W C |
| | 13M | C R S H W D T W C |
| | 2EM | C R S H W D T W C |
| | 3EM | C R S H W D T W C |
| | 4EM | C R S H W D T W C |
| | 6EM | C R S H W D T W C |
| | 11EM | C R S H W D T W C |
| S4 | 1M2 | C S V W D T S G W C |
| | 6M2 | C S V W D T S G W C |
| | 7M2 | C S V W D T S G W C |
| | 9M2 | C S V W D T S G W C |
| | 10M2 | C S V W D T S G W C |

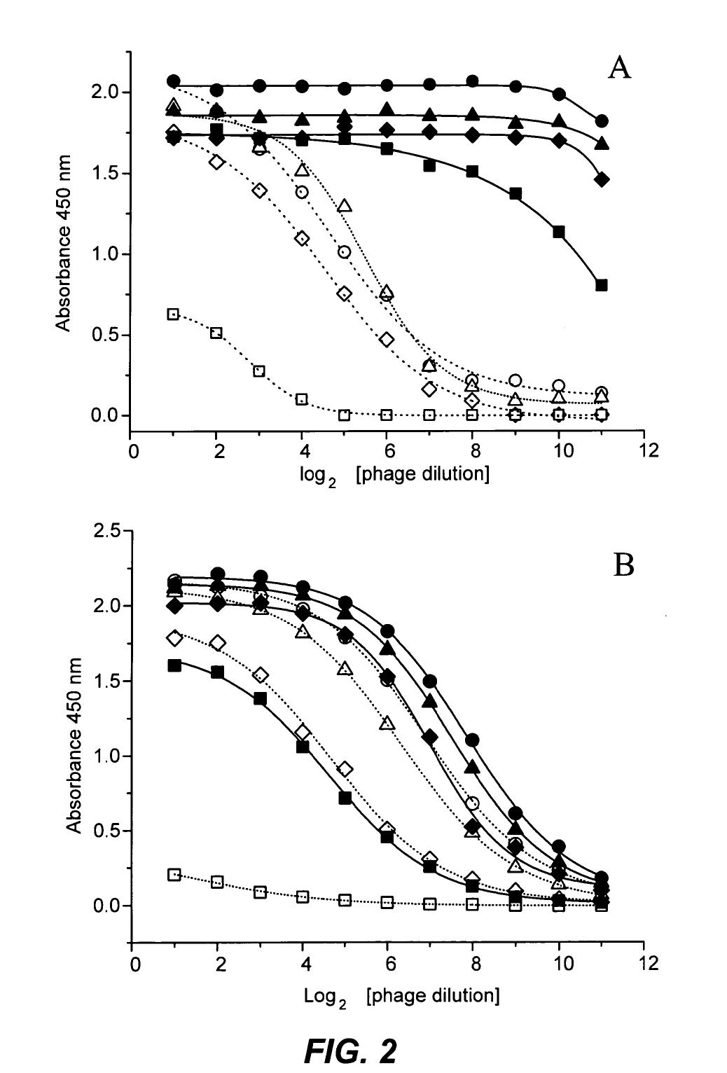

Indeed, not all selected clones performed identically in noncompetitive immunoassays. To eliminate peptides that bind to unbound antibodies, the candidate phage borne peptides were further selected by check board titration. FIG. 2 shows the titration curves of clones 8M2 and 10M2, bearing the S2 and S4 sequence respectively, in the presence or absence of molinate, when different amounts of MoAb 14D7 were used for coating. Notice that in a certain range of phage dilutions, there was saturated binding of the phage in the presence of the analyte, and no binding in its absence (FIG. 2A). Phage clones with the S1 sequence behaved similarly. This range of dilutions shifts according to the amount of antibody used for coating, and constitutes the practical window in which each phage clone can be used for the set up of noncompetitive assays. Not all clones had the same performance, and as shown in FIG. 2B for clone 10M2, no such window was observed, and only at the lowest density of coating and high concentration of phage was it possible to observe significant differential binding. Phage clones with the S3 sequence behaved similarly.

### Optimization of Noncompetitive ELISAs for Molinate

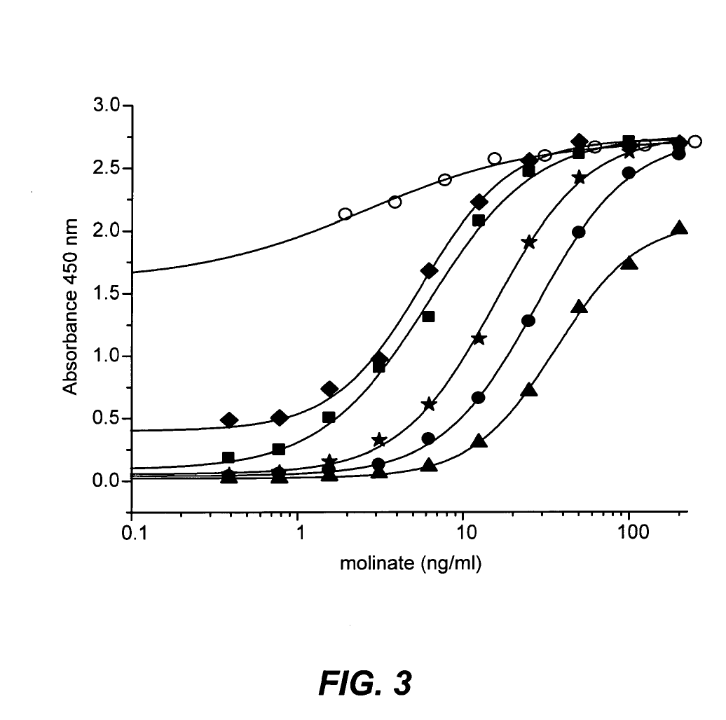

Clone SM2, bearing the S2 sequence, was selected for further assay development. Preliminary experiments with the noncompetitive format indicated that low amounts of coating antibody and high concentrations of phage particles provided the highest assay sensitivities. However, once the antibody coating density has been fixed, the assay sensitivity is determined by the highest phage concentration that produces an acceptable background noise (FIG. 3). Under these conditions, high assay sensitivity was obtained.

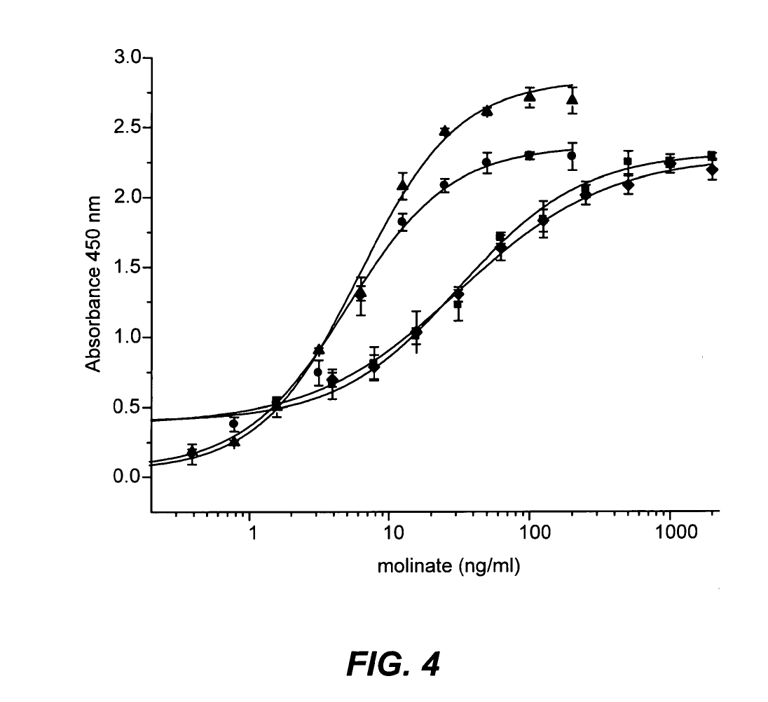

In a similar way that the IC₅₀ (the analyte concentration producing 50% inhibition) is used to denote the sensitivity of competitive methods, we refer to the concentration of analyte producing 50% saturation (SC₅₀) in noncompetitive assays as a practical parameter to compare the sensitivity of our assays. FIG. 4 shows the performance of four different noncompetitive assays set up with each of the different peptide sequences isolated in the panning experiments. For each of these clones, the assay conditions were optimized as described previously. The phage clones with lower residual reactivity with the uncombined antibody showed the best performance, with low background readings and the highest sensitivity. These differences highlight the importance of the residues that flank the WDT consensus sequence of the selected peptides. This can be seen in the case of the S1 and S4 peptide sequences, which only differ in two amino acids and yield assays with SC₅₀ of 5 and 32 ng/ml, respectively. Also small sequence differences between S1 and S2 resulted in higher readings for the latter, which may indicate stronger binding. These observations suggest that the construction of mutagenesis libraries design on the basis of the consensus sequence obtained from naive libraries may allow further improvement of the noncompetitive assay.

### Analysis of Cross Reactivity

The set up of noncompetitive immunoassays using peptides loops as devised in this work relies on the specific reactivity of a short peptide loop with the modify surface of the antigen binding site of the antibody upon binding of the analyte. To test whether the binding of the peptide could stabilize the non-specific interaction of analyte related molecules with the antibody, we assessed the cross reactivity of the assay using a panel of thiocarbamate herbicides. As observed in FIG. 4, only slight cross reaction with a few compounds was detected. The cross reactivity pattern was similar for the two clones assayed and comparable to that of the competitive assay (Rufo et al., J Agric Food Chem, 52(2): p. 182-7 (2004)).

### Use of Phage Borne Peptides to Set Up a Rapid Method for the Detection of Molinate in Water

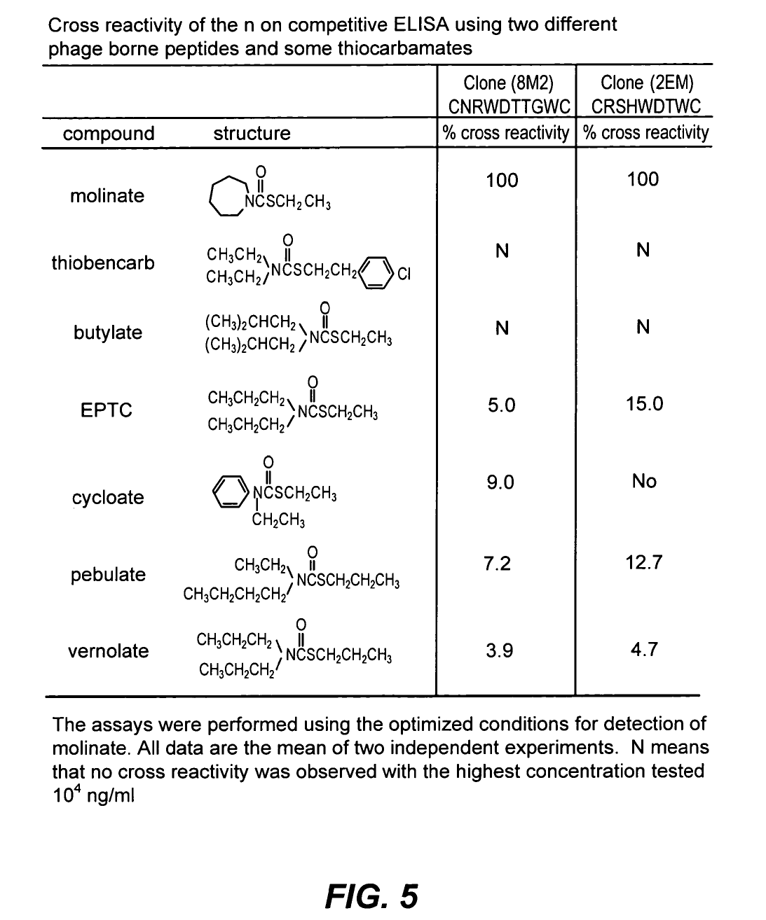

One of the most attractive features of the noncompetitive methods is the possibility of their adaptation into rapid test formats, where the presence of the analyte can be visualized in a yes or no fashion, without the aid of any special instrumentation. To explore this possibility, clone 8M2 was biotinylated and used to set up a dipstick assay. For this, decreasing amounts of MoAb 14D7 were immobilized on nitrocellulose strips. The strips were incubated with different concentrations of molinate, followed by incubation with an appropriated dilution of the biotinylated phage suspension and detection with streptavidin-alkaline phosphatase (FIG. 5). Adjusting the amount of immobilized antibody is was possible to obtain a yes or no assay. Using (2 ng/spot) the presence of 20 ng/ml of molinate could be easily detected by visual inspection. This is the threshold limit for this compound in drinking water in the State of California (see, e.g., the website for OEHHA at oehha.ca.gov/public_info/public/phg4.html. (1988)).

### Conclusions

Using the MoAb 14D7-molinate system, a selection process was devised that made possible the isolation of small peptide loops from phage display libraries, which could be used to detect the formation of the immunocomplex in noncompetitive assay formats. These phage immunoassays not only produce a signal that is proportional to the concentration of analyte, but they also performed with higher sensitivity than the competitive assay set up with heterologous chemical haptens (IC₅₀=69 ng/ml (Rufo, C. et al., J Agric Food Chem, 52(2): p. 182-7 (2004))) or than the phage competitive assay (IC₅₀=45 ng/ml (Cardozo, S. et al., Environ Sci Technol, 39(11): p. 4234-41 (2005))). In addition, they are more suitable for adaptation into rapid tests.

### Example 2

### Development of a Noncompetitive Immunoassay to Detect Atrazine

In this case, we used the MoAb K4E7 specific for atrazine. The conventional chemical hapten assay developed with this MoAb (see, e.g., Giersch, J Ag. Food Chem. 41: 1006-1011 (1993)) allows one to reach an IC₅₀ of 0.5 ng/ml. We used the atrazine-MoAb K4E7 immunocomplex to select phage borne peptides that could be used to develop a noncompetitive assay. The protocols used for panning and assay set up were identical to those used for the molinate-MoAb 14D7 system described in Example 1 above. Table 2 shows the various sequences isolated with the atrazine-MoAb K4E7 complex. A short consensus sequence was identified: WFD. The consensus sequence can occur at position two or five of the random sequence. The only exception is clone 12A which has a three residue stretch after the first cysteine that resembles the WFD consensus sequence, where another acidic residue substitutes for the aspartic acid residue of the consensus. All these clones showed strong reactivity with the immunocomplex, showing that the isolation of peptides that bind to the molinate-14D7 antibody complex is not an isolated phenomenon associated with this system.

TABLE 2

Peptides that bind to the atrazine-K4E7
antibody complex

| | |
|---|---|
| Clone | sequence |

| | |
|---|---|
| 14A | CRWFDNSMLC |
| 4A | CSWFDEGGLC |
| 7A | CYWFDAPSAC |
| 9A | CPSSRWFDLC |
| 11A | CMWFDAYAAC |
| 13A | CTPVRWFDMC |
| 12A | CWHEALTGAC |

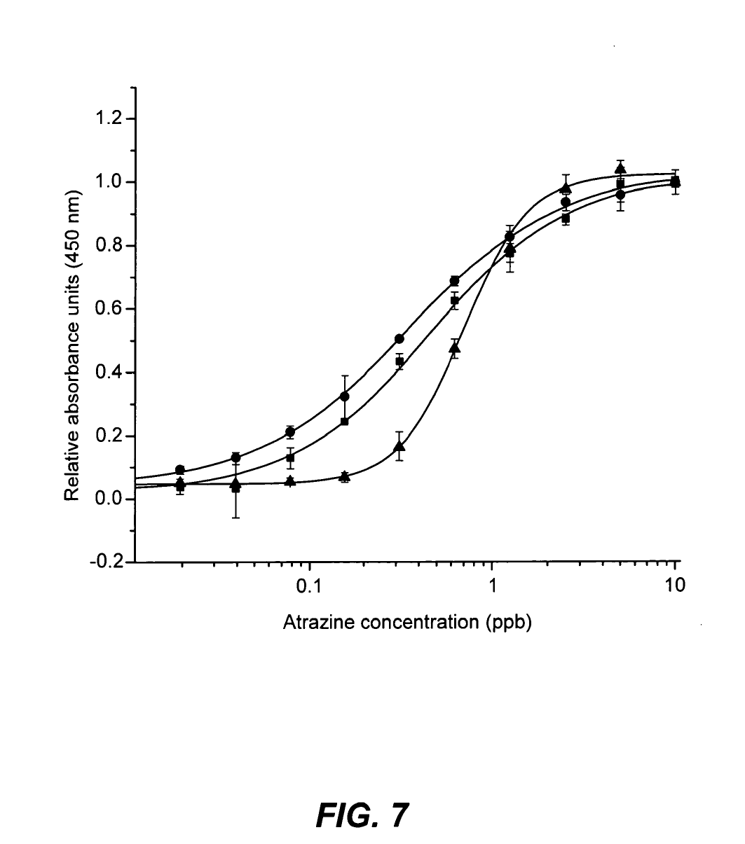

Three of these clones (14A, 4A and 12A) were prepared as stabilized phage suspensions, and the antibody and phage concentration optimized by checkerboard titration as described for the molinate-MoAb 14D7 system. Under these conditions, the noncompetitive assays shown in FIG. 7 were developed.

Again, it was possible to set up sensitive noncompetitive assays for atrazine. The SC₅₀ in this case was similar to that of the conventional chemical hapten assay (0.5 ng/ml) and ranged from 0.2-0.8 ng/ml. As in the molinate-MoAb 14D7 system, the flanking residues of the consensus sequence also appeared to affect assay sensitivity, which reinforces the hypothesis that it should be possible to improve the reactivity of the peptides with the immunocomplex by panning of mutagenesis libraries.

### Example 3

### Further Development of a Noncompetitive Immunoassay to Detect Molinate and Atrazine

### A. Methods and Materials

### Antibodies and Herbicide Related Compounds

Molinate and the thiocarbamate compounds used in the cross-reactivity assays were obtained from Stauffer Chemical Co. Thiobencarb was obtained from Chevron Chemical Co. Development of the monoclonal anti-molinate antibody (MoAb 14D7) has been described in detail previously (see, e.g., Rufo, C., et al., Robust and sensitive monoclonal enzyme-linked immunosorbent assay for the herbicide molinate. J Agric Food Chem, 2004. 52(2): p. 182-7). Atrazine and relate triazines were obtained from Dr. Shirley Gee, and the anti-atrzine antibody was obtained form Dr. T. Giersch (see, e.g., Giersch, T., A new monoclonal antibody for the sensitive detection of atrazine with immunoassay in microtiter plate and dipstick format. Journal of Agriculture and Food Chemistry, 1993. 41: p. 1006-1011).

### Phage Display Peptide Libraries

The phage display peptide library used for initial panning experiments were obtained from Affymax Research Institute, Palo Alto, Calif. This is a constrained library constructed in the phagemid vector p8V2 (see, e.g., Wrighton, N. C., et al., Small peptides as potent mimetics of the protein hormone erythropoietin. Science, 1996, 273(5274): p. 458-64), expressing 7 and 8 random amino acid peptides flanked by two cysteine residues and linked to the N-terminus of the major pVIII phage coat protein through a long glycine-rich spacer (GG-C(X)_{7/8}C(GGGGS)₃—). The library size is 2.4×10⁹ independent clones. For mutagenesis library construction, 200 μg of p8V2 were digested with the BstXI restriction enzyme and the digested vector was purified by ultracentrifugation on a potassium acetate gradient at 48,000 rpm. The ethidium bromide visualized band corresponding to linearized p8V2 vector was separated; the dye removed with butanol, and the DNA precipitated with ethanol. Double stranded DNA coding for the randomized eight amino acid constrained peptides was constructed by annealing of 100 picomoles of oligonucleotide mol mut 1 (5′-GGCCCAGTGCTCACGCAGGAGGCTGTNNKNNKTGGGAHACNNNK TGTGGAGGCGGGGGTAGC-3′) and 100 picomoles of oligonucleotide ON-2 (5′-TAGGGCCCACCTTGCTGGGATCGTCACTTCCC CCACCGCCGCTACCCC CGCCTCC-3′) in 40 mM Tris-HCl, pH 7.5, 20 mM MgCl₂, 50 mM NaCl in a final volume of 45 μl. After heating for 5 min at 70° C. and slowly cooling to room temperature to allow annealing, a fill-in reaction was performed by adding 80 units if sequenase V 2.0 (Stratagene) and 200 μM of dNTPs. After incubation at 37° C. for 1.5 h, excess dNTPs were removed by gel filtration using Microspin S-200 HR columns (Pharmacia). The purified double stranded DNA was digested with BstXI and BsiHKAI by incubating overnight at 55° C. and the digested fragment was purified by gel filtration using Microspin S-300 HR columns (Pharmacia). Twenty micrograms of digested p8V2 were ligated with 1.25 μg of the dsDNA insert using T4 DNA ligase (InVitrogen). The ligated DNA was precipitated with ethanol and dissolved in 20 μl of water. ARI 236 cells (Affymax Research Institute, Palo Alto, Calif.) were electroporated, transferred to SOC medium and incubated without selection for one hour at 37° C. Dilutions of a small aliquot of cells were plated on LB agar petri dishes with ampicillin, allowing an estimation of library diversity of 10⁸ independent clones. Cells were diluted in 500 mL SOP medium (2% Bacto tryptone, 1% Bacto yeast extract, 100 mM NaCl, 15 mM K₂HPO₄, 5 mM MgSO₄, pH 7.2 with NaOH) containing 2.5 ml 20% glucose, 1.5 ml and 50 mg/ml ampicillin and grown at 37° C. with vigorous shaking. After the culture reached an OD₆₀₀=0.5, 10¹² transducing units of helper phage were added and incubated without shaking for 30 minutes to allow phage infection. After that, 0.6 ml of 20 mg/ml kanamycin and 6 ml of 20% arabinose were added to the culture and phages were cleared the next day as described below.

### Biopanning

For the selection procedure, Nunc-Immuno™ plates were coated with 100 μl of MoAb 14D7 (2 μg/ml) in phosphate-buffered saline (PBS) overnight at 4° C. After a blocking step of 1 hour at 37° C. with 5% skimmed milk in PBS, 100 μl of 1000 ng/ml of molinate was added to form the molinate-MoAb 14D7 immunocomplex. After 20 min incubation, the wells were rinsed twice with cold PBS, then 100-1000 library equivalents (≅10¹⁰ transducing units) were diluted in 600 μl of 5% skimmed milk in PBS containing 100 ng/ml of molinate, dispensed into 6 microtiter wells and incubated for 2 hours at 4° C. Unbound phages were removed by 20 washes with cold PBS containing 0.05% Tween 20 (PBST). Bound phages were eluted by a 10 min incubation with 0.1 N glycine-HCl, pH 2.5, 0.1% BSA, and immediately neutralized with 2 M Tris base. The eluted phage was directly amplified as described below; or alternatively, a post-selection-adsorption step was included in some panning experiments to deplete the eluted phage of clones with affinity for the uncombined antibody. For this step, the eluted phage was incubated for 30 min in ELISA wells coated with MoAb 14D7 in the absence of molinate. These phage preparations (600 μl) were added to 10 ml of log-phase E. coli ARI 292 cells (Affymax Research Institute, Palo Alto, Calif.) and amplified in Luria-Bertoni (LB) media containing 0.25% K₂HPO₄, 0.1% MgSO₄, 0.1% glucose and 100 μg/ml ampicillin to an OD₆₀₀=0.4. M13KO7 helper phage (Pharmacia Biotech) at a multiplicity of infection 10:1 was added. After a period of 30 min at 37° C. without shaking, arabinose and kanamycin were added to a final concentration of 0.02% and 40 μg/ml respectively, and the cultures incubated overnight at 37° C. with vigorous shaking. Phage from liquid cultures were obtained by clearing the supernatants by centrifugation at 12,000×g for 15 min, precipitated with 0.2 volumes of 20% polyethylene glycol 8000-2.5M NaCl, (PEG-NaCl) on ice for 1 hour, and centrifuged as above. Phage pellets were resuspended in 2 ml of sterile PBS and titrated in ARI 292. A number of 10¹⁰ transducing units were used for the next round of selection.

After three to four rounds of panning, serial dilutions of individual amplified phage clones were tested for their ability to bind to the molinate-antibody immunocomplex by phage ELISA. Positive clones were further selected by check board titration and submitted for DNA sequencing using the primer ON891 (tgaggcttgcaggggtc) (Division of Biological Sciences, Automated DNA Sequencing Facility, UCDavis).

### Phage ELISA

Nunc-Immuno™ Maxi-Sorp™ plates were coated with MoAb 14D7 or molinate-MoAb 14D7 immunocomplex as described previously. The wells were blocked with 5% skimmed milk PBS, and washed three times with PBST. One hundred μl/well of an overnight culture of individual amplified phage clones were dispensed into the wells. The microtiter plates where then incubated for 1 h at room temperature with gentle rocking. After washing three times with PBST, 100 μL of a 1/5000 dilution of horse radish peroxidase (HRP) labeled anti-M13 monoclonal antibody (Pharmacia, Uppsala) in PBST were added to each well. One hour later, the plates were thoroughly washed and 100 μl of peroxidase substrate (25 ml of 0.1 M citrate acetate buffer pH 5.5, 0.4 ml of 6 mg/ml DMSO solution of 3,3′,5,5′-tetramethylbenzidine, and 0.1 ml of 1% H₂O₂) were dispensed into each well. The enzymatic reaction was stopped after 15-20 min by the addition of 50 μl of 2 M H₂SO₄, and the absorbance at 450 nm (corrected at 600 nm) was read in a microtiter plate reader (Multiskan MS, Labsystems). The supernatants showing high readings in wells coated with the immunocomplex and low response in antibody coated wells were prepared as stabilized phage suspensions (see below), and use for further analysis. For checkerboard titration, 100 μl of variable concentrations of the antibody (0.3-2 μg/ml) were used for coating.

### Stabilization of Phage Suspensions

Individual amplified phage clones were obtained as described above. After two steps of precipitation with PEG-NaCl, the phage particles were suspended in 1/50 volume of the original culture volume in PBS, which was supplemented with Complete Protease Inhibitor Cocktail (Roche Diagnostics) and sodium azide 0.05%. The preparations were filtered through a 0.22 μm filter and stored in aliquots at 4° C. and −20° C.

### Noncompetitive ELISA.

Fifty μl per well of serial dilutions of molinate standard in PBST were dispensed into microtiter plates coated with MoAb 14D7, followed by the addition of 50 μl/well of the adequate dilution of the stabilized phage suspension. The antibody concentration used for coating and the proper dilution of the phage suspension had been previously optimized by checkerboard experiments. After an hour incubation period at room temperature, the plates were washed and developed as previously described. Absorbance values were fitted to a four-parameter logistic equation using the Genesis Lite 3.03 (Life Sciences (UK) Ltd.) package software. The lower detection limit (DL) of the assays was estimated as the analyte concentration corresponding to the absorbance equal to that of the zero standard plus three times its standard deviation).

### Assay Cross-Reactivity

The specificity of the noncompetitive assay set up with the phage borne peptides was characterized by determining the cross-reactivity with related pesticides. Molinate concentrations in the 0-1,000 ng/ml range were used in the noncompetitive ELISA. After the data were normalized, the molar compound concentration corresponding to the midpoint of the curve, which corresponds to the concentration of analyte producing 50% saturation of the signal (SC₅₀), was used to express the cross-reactivity of the assay according to the equation:

% cross−reactivity=100×[SC₅₀(molinate)/SC₅₀(cross−reacting compound)]

### Dipstick Assay

One and a half μl of serial dilutions of MoAb 14D7 in PBS were spotted onto nitrocellulose membranes 0.45 microns (Biorad Laboratories Inc., CA). Immediately after drying, the membranes were quenched by soaking into 5% skimmed milk in PBS for 30 min; after rinsing in PBST, the membranes were cut into strips, dried and kept at 4° C. until used. Five hundred μl of a stabilized phage suspension (about 10¹³ phage particles/ml) were dialyzed overnight at 4° C. against 100 mM NaHCO₃, pH 8.5 buffer, and biotinylated by incubation with biotinamido-caproate N-hydroxysuccinimide (Sigma, Ill.), followed by extensive dialysis against PBS. The biotinylated phage suspension was supplemented with Complete Protease Inhibitor Cocktail (Roche Diagnostics), 0.05% sodium azide, filtered through a 0.22 μm filter and stored in aliquots at 4° C. and −20° C. until used. The assay was performed by dipping the membrane strips for 15 min into water spiked with different amounts of molinate and containing the appropriate dilution of biotinylated phage, washed under tap water, incubated for 10 min in a diluted solution of streptavidin-HRP, and developed using the diaminobenzydine-NiCl₂ substrate.

### B. Results and Discussion

Antibody-Analyte IC Specific Peptides can be Isolated from Phage Display Peptide Libraries

The selection of the library was performed on plates coated with the molinate-MoAb 14D7 complex in the presence of 100 ng/mL of molinate. After three rounds of panning individual clones were grown and assayed for differential reactivity with the IC and the unreacted antibody. Table 3 shows the peptide sequences of 20 clones exhibiting the biggest differences. 4 different sequences were obtained, three of them from the 8-mer library. All had the consensus sequence WDT, shown in bold in the table. While this motif seemed to be important for binding to the antibody-analyte complex, the flanking residues also played a significant role in peptide performance, particularly with regard to the residual reactivity of the peptide with the uncombined binding sites of the antibody. This was evident when check-board titration analysis was used to optimize the amount of coating antibody in the presence of a fix amount of analyte. In general, the biggest differences in the reactivity of the phage borne peptide with the free antibody and the IC were observed at low antibody coating density and high phage dilutions. This was particularly true for clones 2EM and 10M2 (not shown).

TABLE 3

Peptide sequences isolated with the
molinate-MoAb 14D7 immunocomplex

| | |
|---|---|
| Clone | sequence |

| | | |
|---|---|---|
| 1M | C S T W D T T G W C(2) | |
| 8M2 | C N R W D T T G W C(5) | |
| 2EM | C R S H W D T W C | (8) |
| 10M2 | C S V W D T S G W C(5) | |

The number of isolates bearing the same sequence is indicated between brackets.

### Set Up of Noncompetitive Phage Anti-Immunocomplex Assay (PHAIA) for Molinate

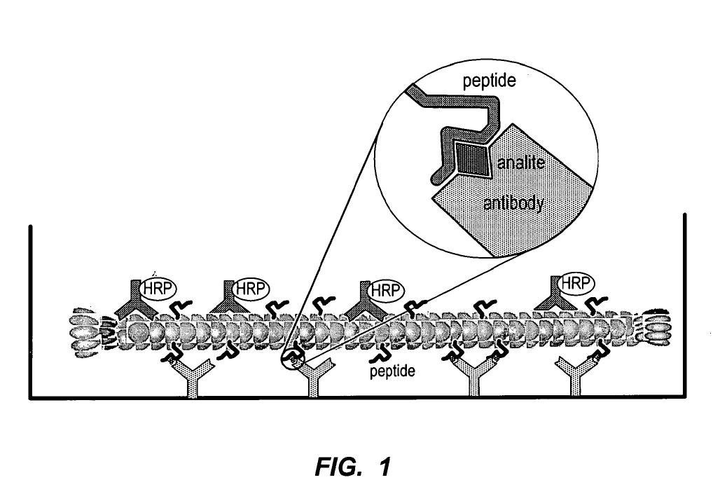

Once the amount of antibody was fixed, we developed a noncompetitive format, PHAIA, to measure the analyte-bound sites using the set up shown in FIG. 1. There was a direct correlation between measured signal and amount of analyte in a wide range of molinate concentrations. In general, the sensitivity of the PHAIA increased with the concentration of phage particles use in the assay. However after certain point, this also increased the background noise of the assay, most probably due to direct binding of the peptide to the uncombined antibody (FIG. 3A). The assay performance was influenced by the sequence of the phage borne peptides used for IC detection (FIG. 4). The phage clones with lower residual reactivity with the uncombined antibody showed the best performance (1M and 8M2), exhibiting low background readings and the highest sensitivity. In a similar way that the IC₅₀ (the analyte concentration producing 50% inhibition) is used to denote the sensitivity of the competitive methods, the midpoint of the doses-response curve, which corresponds to the concentration of analyte producing 50% saturation (SC₅₀), was used to compare the sensitivity of the assays. The most sensitive test was attained with clone 1M. With this phage borne peptide, the DL was 0.73 ng/ml, and the SC₅₀ 5.0±0.4 ng/ml. This sensitivity is about 14 times that of the competitive ELISA, IC₅₀=69.2±1.4 ng/ml as reported by Rufo et al. (see, e.g., Rufo, C., et al., Robust and sensitive monoclonal enzyme-linked immunosorbent assay for the herbicide molinate. J Agric Food Chem, 2004, 52(2): p. 182-7). Considering that the other three clones also performed with better sensitivity than the competitive assay, and that the same MoAb was used in both ELISAs, this result shows the utility of anti-IC phage borne peptides for immunoassay development.

In order to increase the repertoire of candidate peptide ligands for the IC and evaluate the role of the flanking residues of the WDT motif, an 8-mer mutagenesis library was constructed. This library had the general structure CXXwdtXXXC, where X indicates a fully random position, and lowercase letters indicate a 50% probability of finding this residue in that position. Different strategies were used to pan this library, including long washing steps that would favor the selection of high affinity binders, and adsorption steps of the neutralized phage pool eluted from the IC on fresh uncombined antibody. Many additional sequences could be isolated in this way that are summarized in Table 4. All sequences isolated from the 8-mer mutagenesis library contained the consensus S/N X W D T T/S G W, where in addition to the WDT motif, Gly and Try were found in position 7 and 8, respectively. Position 2 showed a degree of variation, but even there, there was a prevalence of small hydrophobic residues. Position 6, which was occupied by Thr or Ser, appeared to have a strong influence on assay sensitivity, and in the analyzed clones, peptides showing low SC₅₀ have Thr in this position.

TABLE 4

IC-reactive peptide sequences isolated from
the naïve and mutagenesis library

| | | |
|---|---|---|
| Clone | sequence | SC₅₀ (ng/ml) |

| | | |
|---|---|---|
| 1M | C S T W D T T G W C | 5.0 ± 0.4 |
| 8M2 | C N R W D T T G W C | 6.0 ± 0.3 |
| mI12 | C S I W D T T G W C | 8.1 ± 0.6 |
| mA | C S L W D T T G W C | 8.5 ± 0.5 |
| mD | C S V W D T T G W C | ND |
| mF | C N L W D T T G W C | 11 ± 0.5 |
| mE | C N P W D T T G W C | 16 ± 0.5 |
| mG | C S Y W D T T G W C | 16 ± 0.7 |
| mH | C N I W D T S G W C | 27 ± 2.0 |
| 10M2 | C S V W D T S G W C | 32 ± 4.9 |
| mE | C S L W D T S G W C | ND |
| mC | C H I W D T S G W C | ND |
| mI13 | C N V W D T S G W C | ND |

The clones isolated from the mutagenesis library are denoted by an initial “m” in their names.

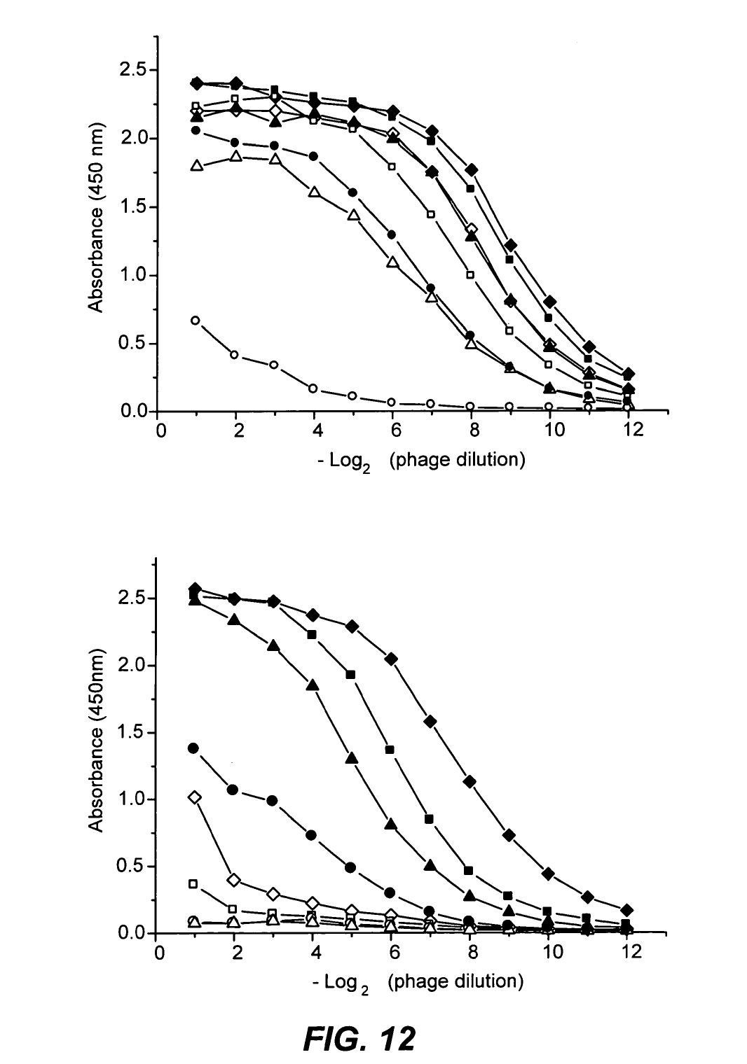

As mentioned above, in addition to its influence on assay sensitivity, the peptide sequence also influenced its residual reactivity with the free antibody. In order to minimize this feature, we biased the panning experiments of the mutagenesis library by including post-adsorption steps on the uncombined antibody. Consequently, most of the clones isolated from this library showed lower residual binding to MoAb 14D7 than the original clones selected from the naíve library. This is exemplified in FIG. 12, where clone mA, selected from the mutagenesis library, is compared with clone 1M. While clone 1M requires limited amounts of coating antibody to minimize its binding to the uncombined antibody, clone mA showed little reactivity with the free antibody in a wide range of antibody coating densities. However, this intuitive advantage of clone mA (which is shared by other clones isolated from the mutagenesis library) was not directly translated into a more sensitive test as can be seen in Table 4. This highlights the relevance of the overall affinity of the peptide for the IC, as a feature that determines the sensitivity of the assay. This overall affinity results from the combined interactions of the peptide with the exposed regions of the analyte plus additional binding to residues of the antibody. While the latter may be the source of cross-reactivity with the free antibody, it also works to improve the detection of trace amounts of IC in the PHAIA system.

### The Capture Antibody Determines the Specificity of the PHAIA Method

The set up of noncompetitive immunoassays using peptides loops as devised in this work utilizes the specific reactivity of a short peptide loop with the modified surface of the antigen binding site of the antibody upon binding of the analyte. To test whether the binding of the peptide could stabilize the non-specific interaction of analyte related molecules with the antibody, we assessed assay cross reactivity using a panel of molinate-related thiocarbamate herbicides. As observed in FIG. 4, only slight cross reactivity with a few compounds was detected. The cross reactivity pattern was similar for the two clones examined (1M (8-mer) and 2EM (7-mer)) and comparable to that of the competitive assay set up with the same antibody (see, e.g., Rufo, C., et al., Robust and sensitive monoclonal enzyme-linked immunosorbent assay for the herbicide molinate. J. Agric. Food Chem, 2004, 52(2): p. 182-7), indicating that the capture antibody has a distinct influence on assay cross reactivity.

### Adaptation of PHAIA to Dipstick Formats

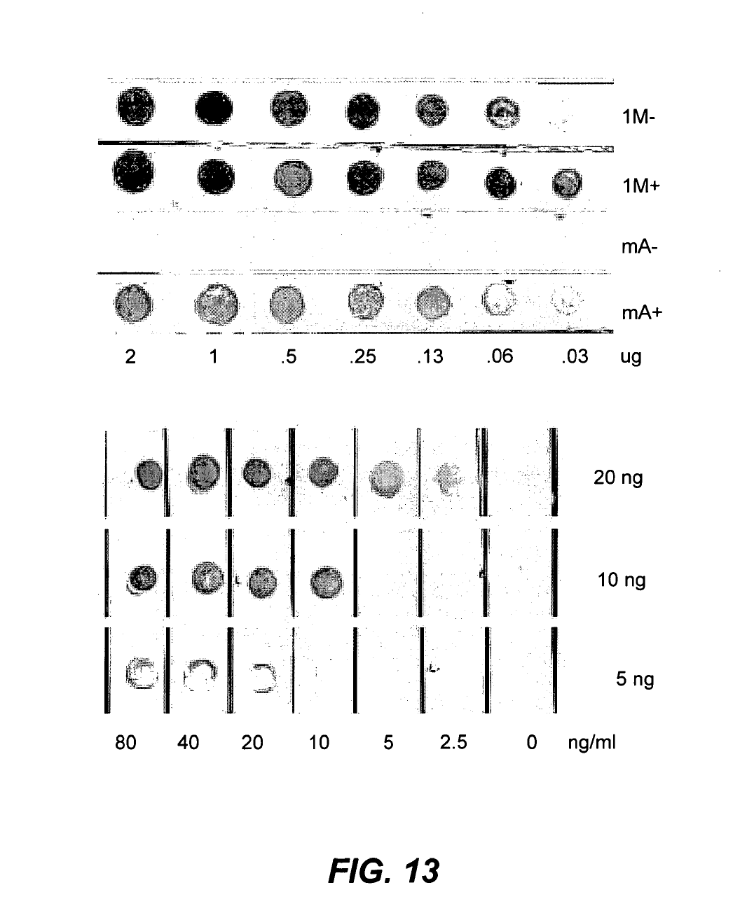

One of the most attractive advantages of noncompetitive assays is the possibility of measuring near-zero signals at low analyte concentration. This is a key feature that explains the improved sensitivity of noncompetitive assays over competitive ones, but it is also an advantageous property that facilitates their adaptation into rapid test formats, particularly, when the presence of the analyte needs to be visualized in a yes or no fashion. To accomplish this goal, we adapted the assay into a dipstick format where the antibody immobilized onto a nitrocellulose strip is dipped into the analyte solution and then the formation of the IC is revealed by its reaction with the phage borne peptide. This reaction could be visualized either by direct labeling of the phage particles or with the use of a secondary antibody. Again, the residual reactivity of the peptide with the uncombined antibody played a role in establishing a robust assay. As shown in FIG. 13, there was no background reactivity in a wide range of antibody coating densities for clone mA, but clone 1M required careful adjustment of the amount of capture antibody. However, once the proper conditions were set, clone 1M could be used to detect up to 2.5 ng/ml of molinate by visual examination. This is a remarkable result considering that the WHO guideline value for the concentration of this compound in drinking water is 6 ng/ml (see, e.g., Hamilton, D., et al., Regulatory limits for pesticide residues in water. Pure Appl. Chem., 2003. 75(8): p. 1123-1155), and 20 ng/ml in the State of California (see, e.g., the website for OEHHA at oehha.ca.gov/public_info/public/phg4.html. (1988)).

### The Use of Peptides for IC Detection can be Extended to Other Analyte-Antibody Systems

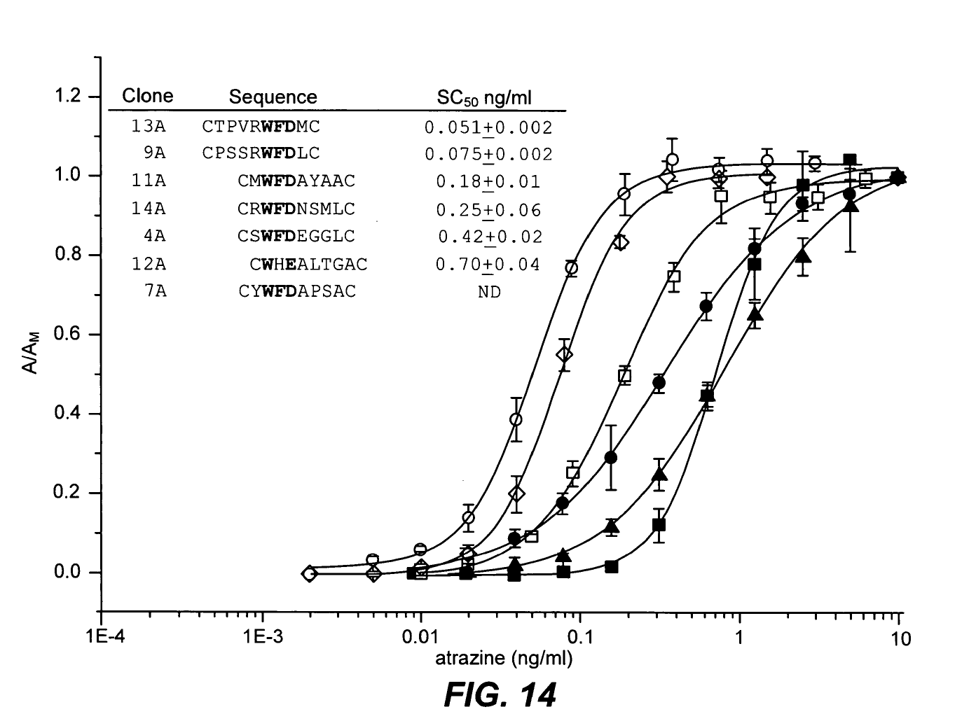

The isolation of peptides that exhibited a differential reactivity with the antibody in the presence or absence of the small molecule analyte constitutes one aim of PHAIA technology. To establish the applicability of this technology to other analyte-antibody systems, we explored the adaptation of the competitive immunoassay to the herbicide atrazine described by Giersch (see, e.g., Giersch, T., A new monoclonal antibody for the sensitive detection of atrazine with immunoassay in microtiter plate and dipstick format. Journal of Agriculture and Food Chemistry, 1993, 41: p. 1006-1011). This is a highly sensitive assay (IC₅₀=0.5 ng/ml) that utilizes the MoAb K4E7. Using the panning strategy describe above, several peptide sequences highly specific for the atrazine-MoAb K4E7 IC were isolated (insert in FIG. 14). All these clones exhibited strong reactivity with the immunocomplex, showing that the isolation of peptides that bind to the molinate-14D7 antibody complex is not an isolated phenomenon associated with this assay system. Once more, there was an evident consensus motif, WFD, that occurred in position 2 or 5 of the random 8-mer peptide. The only exception was clone 12A which has a three residue stretch that resembles the WFD consensus sequence, where another acidic residue substitutes for the aspartic acid residue of the consensus sequence. These peptides allowed the set up of sensitive noncompetitive assays and the corresponding SC₅₀ are summarized in FIG. 14. There was a pronounced influence of the flanking residues of the WFD motif on the reactivity of the peptide. Clone 13A showed the best performance with SC₅₀=0.0511±0.002 ng/ml and DL=0.016 ng/ml. Once more, this represented a significant improvement with regard to the competitive assay set up with the same antibody (see e.g., Cardozo, S., et al., Analyte peptidomimetics selected from phage display peptide libraries: a systematic strategy for the development of environmental immunoassays. Environ Sci Technol, 2005, 39(11): p. 4234-41), with IC₅₀=0.64±0.06 ng/ml and DL=0.18 ng/ml. The specificity of the assay was analyzed using triazine-related herbicides (Table 5). Similarly to the molinate-MoAb 14D7 system, the specificity was determined by the capture antibody, with a cross-reactivity profile highly similar to that obtained with MoAb 4KE7 using a competitive assay (see, e.g., Cardozo, S., et al., Analyte peptidomimetics selected from phage display peptide libraries: a systematic strategy for the development of environmental immunoassays. Environ Sci Technol, 2005, 39(11): p. 4234-41).

### C. Conclusions

Despite the many advantages of two-site immunoassays, to date, no systematic and simple methods exist for the development of these assays when the analyte is a low molecular weight molecule. The small size of these analytes an obstacle that precludes the simultaneous binding of the capture and detecting antibody. The closest approach to overcome this situation has consisted of the use of antiidiotype antibodies that show differential reactivity with the antibody binding site upon binding of the analyte. However, as discussed above, due to the large surface buried in the antiidiotype IC interface, these antibodies constitute a rare event in the polyclonal immune response against IC antigens, and therefore are difficult to produce. Here we demonstrated that it is possible to use short peptide loops isolated from phage display libraries for trivalent detection of small analytes in a noncompetitive format. In the two models examined above, the transition from detection of unreacted antibody binding sites (competitive assay) towards detection of the analyte-antibody reaction boosted the sensitivity of the tests. The proportional signal of the noncompetitive format allowed the detection of near zero signals, and therefore facilitated the adaptation of the PHAIA into rapid, yet sensitive, dip-stick formats for ‘point of need’ testing.

Another important aspect of PHAIA is its general applicability and simplicity. In addition to the two examples presented here, the concept has also worked for drug analytes and for assays that utilize polyclonal antibodies (as shown in Examples 4 and 5 below). Moreover, we have found that anti-IC peptides can also be isolated from phage libraries expressing different lengths of random peptides, and also from libraries expressing the peptide on the phage minor coat protein pIII. In contrast to the challenging and laborious preparation of detection antibodies, the selection of phage borne peptides is systematic and can be accomplished in a few days using commercially available phage display libraries. Furthermore, the final product of this selection, namely the phage particle bearing the specific peptide sequence, can be directly used as convenient immunoassay reagent (see, e.g., Cardozo, S., et al., Analyte peptidomimetics selected from phage display peptide libraries: a systematic strategy for the development of environmental immunoassays. Environ Sci Technol, 2005, 39(11): p. 4234-41). Moreover, the stability and filamentous polymeric structure of the phage particle open many possibilities for its chemical modification using dyes, fluorescent compounds, acridinium esters, enzymes, etc. which enable the automation of the noncompeptitive PHAIA technology in microfluidics and biosensors platforms.

### Example 4

### Development of a Noncompetitive Immunoassay to Detect Phenoxybenzoic acid (PBA)

### Introduction

In the examples above, we have shown that phages bearing short peptide loops isolated from phage display peptide libraries with hapten monoclonal antibody immunocomplexes (IC) can substitute for anti-IC antibodies and can be used for immunodetection of small analytes. One of the aims of the next two examples is to demonstrate that such phage ligands can be isolated even when the selector ICs are polyclonal in nature. For this demonstration, phenoxybenzoic acid (PBA), a major pyrethroid metabolite, was used as a model system. Panning of an 8-mer cyclic peptide library fused to the pIII phage protein with PBA/anti-PBA IC yielded a peptide sequence specifically recognizing the immune complex. Phages bearing this peptide were tested in a noncompetitive ELISA format, which resulted in a more sensitive assay than the competitive heterologous assay set up with the same antibody. PHAIA was also easily adapted into a rapid and highly sensitive dipstick assay. These results show that PHAIA technology can be readily used to adapt the use of existing polyclonal antibody-based competitive assays into noncompetitive formats. Moreover, this assay technique not only provides a positive reading, but it constitutes a major shortcut in the development of polyclonal based assays by avoiding the need to synthesize the heterologous competing haptens that are usually required to obtain highly sensitive polyclonal immunoassays.

### Materials and Methods

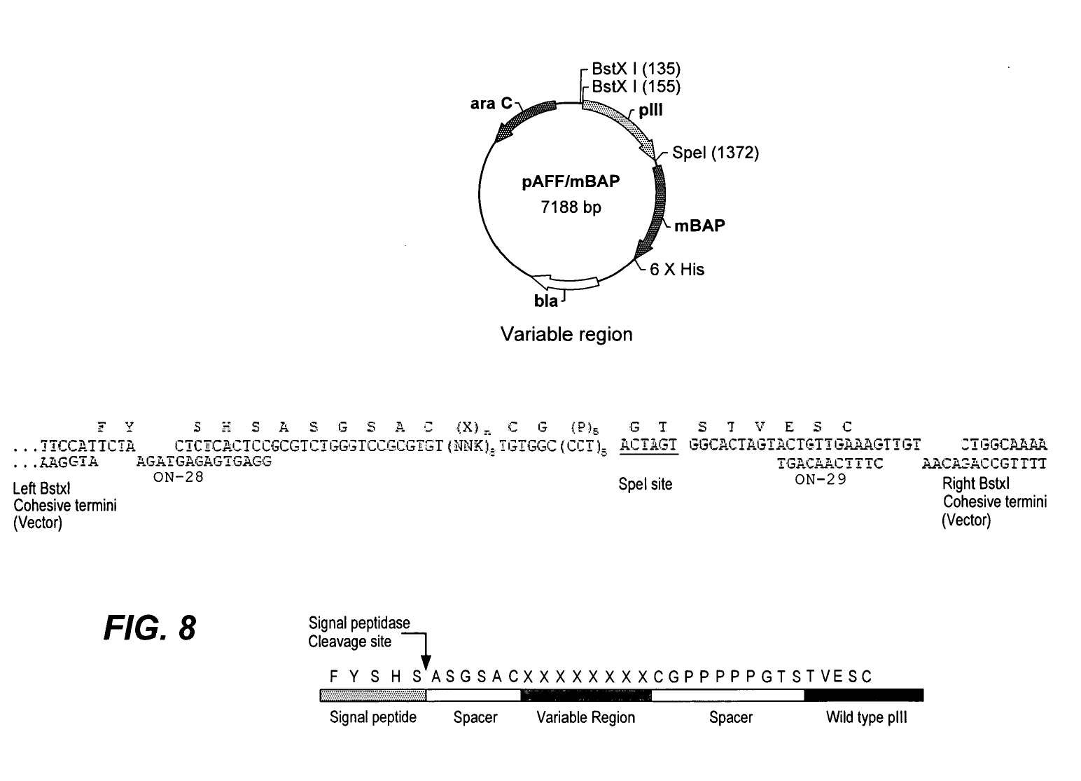

Phage library and antibodies. A peptide phage display library with an estimated diversity of 3×10⁹ independent clones was constructed on the phagemid vector pAFF/mBAP (FIG. 8). This vector has been developed in our laboratory as a modification of the Affymax pAFF2 vector (Martens et al. J Biol Chem 270(36): 21129-36 (1995)), where a mutated form of E. coli alkaline phosphatase (BAP) was introduced to facilitate the production of the peptide-BAP fusion protein after selection of the library. This library expresses cyclic 8-mer random peptides flanked by two cysteines and fused to the phage coat protein pIII. Antibodies specific for PBA, were purified on 3-((2-oxoethoxy)ethoxy)phenoxybenzoic acid coupled Sepharose from serum of rabbits immunized with a hapten-KLH conjugate as described in, e.g., Shan et al., Chem Res Toxicol 17(2): 218-25 (2004).

Panning: Microtiter ELISA plates (Maxisorp, Nunc) were coated with PBA affinity purified antibodies at a concentration of 5 μg/ml in phosphate-buffered saline (PBS) for either 1 hour at 37° C. or overnight at 4° C. After coating, the wells were blocked with 1% BSA at 37° C. for 1 hour and washed three times with PBS, 0.05% Tween 20. The peptide library (10×10¹¹ phage particles) was mixed with PBA (10 μg/ml final concentration) and BSA (final concentration of 1%) in a final volume of 600 μl PBS, and then added to 6 microtiter wells coated with the antibody. After incubating for 2 hours at 4° C., the wells were washed 5 times with PBS, incubated for half an hour in PBS at 4° C., washed again 5 times with PBS, after which, the bound phages were eluted with 100 μl per well of 100 mM glycine-HCl, pH=2.2 buffer. After incubation for 10 min at room temperature, the eluted phage was transferred to a 1.5 ml tube, and the pH neutralized by adding 35 μl of 2M Tris base.

For amplification of the phage stock, the eluted phage (600 μl) were added to 10 ml of log-phase E. coli ARI 292 (Affymax Research Institute, Palo Alto, Calif.) cells and amplified in SOP medium (LB media containing 0.25% K₂HPO₄, 0.1% MgSO₄) plus 0.1% glucose and 100 μg/ml ampicillin to an OD600=0.4. Helper phage M13KO7 (New England Biolabs) at a multiplicity of infection 10:1 was added. After an incubation period of 30 min at 37° C. without shaking, arabinose and kanamycin were added to a final concentration of 0.02% and 40 μg/ml respectively, and the cultures were incubated overnight at 37° C. with vigorous shaking. Phage from liquid cultures were obtained by clearing the supernatants by centrifugation at 12,000×g for 15 min, precipitated with 0.2 volumes of 20% polyethylene glycol 8000-2.5M NaCl, (PEG-NaCl) on ice for 1 hour, and centrifuged as above. Phage pellets were resuspended in 2 ml of sterile PBS and titrated in ARI 292. A number of 10¹⁰ transducing units were used for the next round of selection.

Additional rounds of panning were performed in a similar way, using 200 μl of the amplified phage stock supplemented with BSA (1% final concentration) and PBA at a final concentration of 10 μg/ml.

### Phage ELISA

After three rounds of panning, ARI 292 cells were infected with the eluted phage and grown on LB-Agar Ampicillin plates. Ten individual clones were then picked and used for inoculation of tubes with 5 ml of SOP with ampicillin and glucose as described above, cells were grown with shaking at 37° C. After cultures reached an OD600=0.5 AU, 1 μl of M13K07 helper phage at a concentration of 1×10¹¹ was added for growing individual recombinant phage supernatants. Cultures were then incubated for 30 minutes at 37° C. without shaking for allowing infection of cells. Arabinose and kanamycin were then added as described above and cultures were grown overnight with shaking at 37° C. The next day, the cells were pelleted by centrifugation at 10,000×rpm for 5 minutes and the supernatants used for screening.

ELISA screening for phage that reacted with the PBA-antibody complex was performed by directly adding 50 μl of supernatants and 50 μl of 200 ng/ml of phenoxybenzoic acid (PBA) to microtiter plates coated with 0.5 μg/well of affinity purified anti-PBA polyclonal antibody.

### Stabilization of Phage Suspensions

Individual amplified phage clones were obtained as described above. After two steps of precipitation with PEG-NaCl, the phage particles were suspended in 1/50 volume of the original culture volume in PBS, which was supplemented with (Complete Protease Inhibitor Cocktail) (Roche Diagnostics) and 0.05% sodium azide. The preparation (Master Phage Solution) was filtered through a 0.22 μm filter and stored in aliquots at 4° C. and −80° C.

### Noncompetitive Phage ELISA

ELISA plates were coated with the gamma-globulin fraction of the anti-PBA rabbit serum purified on Protein G columns (Amersham-Pharmacia, Uppsala) using 100 μl of 10, 5, 2.5 and 1.25 μg/ml in PBS. After incubation for 1 hour at 37° C. and blocking for 1 hour at 37° C. with 1% BSA, the plates were washed 3 times with PBS-0.05% Tween 20. Serial 2-fold dilutions of phage (starting from a 1/50 dilution of the Master Phage Solution) were performed in PBS-0.05% Tween 20 in non-treated polystyrene plates (low binding capacity) in the presence (50 ng/ml) of PBA or in its absence. The phage dilutions were then transferred to wells pre-coated with the protein G purified antibody. After incubation for 1 h at 37° C., the plates were washed and 100 μl of the appropriated dilution of an anti-rabbit IgG antibody coupled to peroxidase was added to the wells, incubated for 30 min at 37° C. and thoroughly washed. The peroxidase activity was then developed by adding 100 μl of peroxidase substrate (25 ml of 0.1 M citrate acetate buffer, pH 5.5, 0.4 ml of 6 mg/ml DMSO solution of 3,3′,5,5′-tetramethylbenzidine, and 0.1 ml of 1% H₂O₂) dispensed into each well. The enzymatic reaction was stopped after 15-20 min by the addition of 50 μl of 2 M H₂SO₄, and the absorbance at 450 nm (corrected at 600 nm) was read in a microtiter plate reader (Multiskan MS, Labsystems). Peliminary experiments indicated that coating with 100 μl of 10 μg of antibody and the use of a 1/3200 final dilution of the Master Phage Solution resulted in the biggest ratio of immunocomplex/uncombined antibody reactivity, and this conditions were used to set up the noncompetitive ELISA for PBA.

Panning a Peptide Library with Polyclonal Antibodies Against PBA

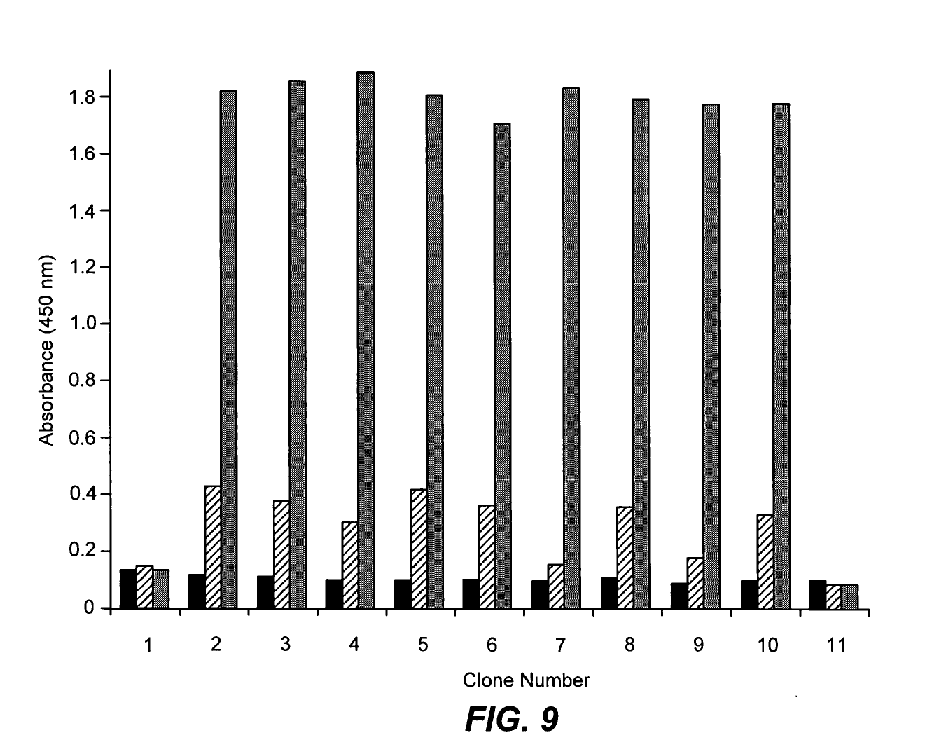

A phage display peptide library expressing peptides of 8 random amino acids flanked by a disulfide bridge and fused to the phage protein pIII was selected as described above. In order to favor the isolation of peptides that recognize the PBA-antibody immunocomplex, an excess of PBA was included during incubation. After 3 rounds of panning, 10 individual clones were tested for binding to the IgG coated wells in the presence or absence of PBA as described above. As shown in FIG. 9, 9 out of 10 clones exhibited stronger binding to the polyclonal antibody in presence of PBA (100 ng/ml, final concentration) than in the absence of it. Plasmids belonging to these 9 clones were sequenced and all of them coded for the same cyclic peptide sequence, C F N G K D W L Y C.

### Noncompetitive ELISA

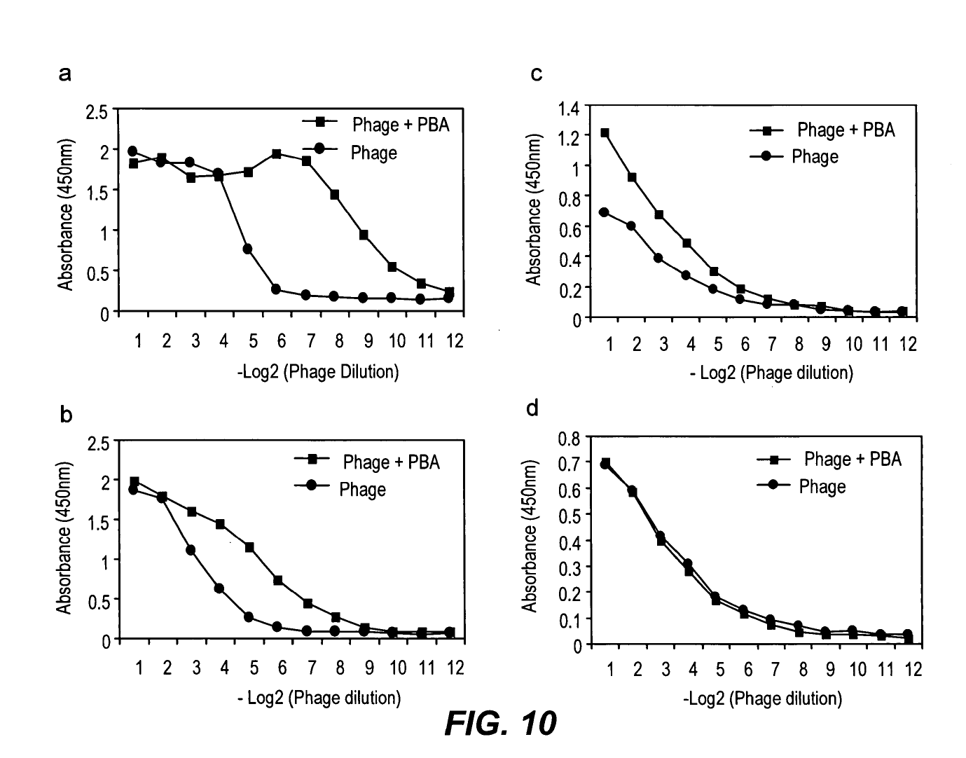

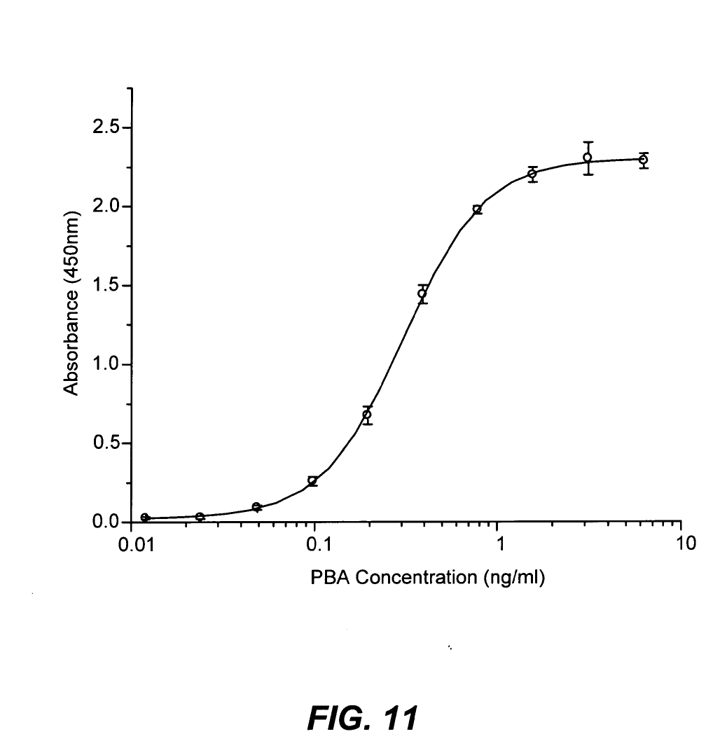

A phage clone displaying the CFNGKDWLYC sequence was amplified and prepared as a stabilized phage suspension as describe above. This phage solution was used to optimize the conditions for coating (FIG. 10). The best results were obtained when 100 μl of 10 μg/ml was used at 1/3200 or higher dilutions of the Master Phage Solution. These conditions were then used to set up the noncompetitive ELISA shown in FIG. 11. The ELISA had a working range of 0.1-1.0 ng/ml and performed with a SC₅₀ of 0.31±0.03 ng/ml (SC=concentration of analyte at which ELISA readings are 50% of the maximal signal). This result represents about a 4-fold improvement in assay sensitivity as compared to the competitive ELISA described in Shan et al., Chem Res Toxicol 17(2): 218-25 (2004) which exhibited an IC₅₀ of 1.29 ng/ml.

### Example 5

### Further Development of a Noncompetitive Immunoassay to Detect Phenoxybenzoic Acid (PBA)

### A. Materials and Methods

### Phage Library and Antibodies

A random phage display peptide library with an estimated diversity of 3×10⁹ independent clones was constructed on the phagemid vector pAFF/MBP as described (see, e.g., Martens, C. L.; Cwirla, S. E.; Lee, R. Y.; Whitehom, E.; Chen, E. Y.; Bakker, A.; Martin, E. L.; Wagstrom, C.; Gopalan, P.; Smith, C. W.; et al. J. Biol. Chem., 1995, 270, 21129-21136). pAFF/MBP vector is a derived vector of the Affymax pAFF2 vector (see, e.g., Martens, C. L.; Cwirla, S. E.; Lee, R. Y.; Whitehom, E.; Chen, E. Y.; Bakker, A.; Martin, E. L.; Wagstrom, C.; Gopalan, P.; Smith, C. W.; et al. J. Biol. Chem., 1995, 270, 21129-21136). This library, with the general sequence ASGSACX₈CGP₆G-, expresses cyclic 8-mer random peptides flanked by two cysteines and fused to the phage coat protein pIII.

Antibodies specific for PBA from serum of rabbits immunized with a hapten-KLH conjugate (see, e.g., Shan, G.; Huang, H.; Stoutamire, D. W.; Gee, S. J.; Leng, G.; Hammock, B. D. Chem. Res. Toxicol., 2004, 17, 218-225) were purified on 3-((2-oxoethoxy)ethoxy)phenoxybenzoic acid-coupled Sepharose. Alternatively, the gamma-globulin fraction of the anti-PBA rabbit serum was purified on Protein G columns as described by the manufacturer (Amersham-Pharmacia, Uppsala), and was used for the ELISA set up.

### Panning

Microtiter ELISA plates (Maxisorp, Nunc) were coated with affinity purified anti-PBA antibodies at a concentration of 5 μg/ml in phosphate-buffered saline (PBS) either for 1 hour at 37° C. or overnight at 4° C. After coating, the wells were blocked with 1% BSA at 37° C. for 1 hour and washed three times with PBS, 0.05% Tween 20 (PBST). The peptide library (10×10¹¹ transducing units) was mixed with PBA (10 μg/ml final concentration) and BSA (final concentration of 1%) in a final volume of 600 μl of PBS, and then added to 6 microtiter wells coated with the antibody. After incubating for 2 hours at 4° C., the wells were washed 5 times with PBS, incubated for half an hour in PBS at 4° C., and washed again 5 times with PBS. Then, the bound phages were eluted with 100 μl per well of 100 mM glycine-HCl, pH 2.2 buffer. After incubation for 10 min at room temperature, the eluted phage was transferred to a 1.5 ml tube and the pH was neutralized by adding 35 μl of 2M Tris base.

For amplification of the phage stock, the eluted phage (600 μl) was added to 10 ml of log-phase E. coli ARI 292 (Affymax Research Institute, Palo Alto, Calif.) cells and amplified in SOP medium (LB media containing 0.25% K₂HPO₄, 0.1% MgSO₄) plus 0.1% glucose and 100 μg/ml ampicillin to an OD₆₀₀=0.4. Then M13KO7 helper phage (New England Biolabs), at a multiplicity of infection 10:1, was added. After a period of 30 min at 37° C. without shaking, arabinose and kanamycin were added to a final concentration of 0.02% and 40 μg/ml, respectively, and the cultures were incubated overnight at 37° C. with vigorous shaking. Phage from liquid cultures were obtained by clearing the supernatants by centrifugation at 12,000×g for 15 min, precipitated with 0.2 volumes of 20% polyethylene glycol 8000-2.5M NaCl, (PEG-NaCl), incubated on ice for 1 hour, and centrifuged as above. Phage pellets were resuspended in 2 ml of sterile PBS and titrated in ARI 292. A number of 10¹⁰ transducing units were used for the next round of selection. Additional rounds of panning were performed in a similar way, using 200 μl of the amplified phage stock supplemented with BSA (1% final concentration) and PBA at a final concentration of 10 μg/ml.

### Phage ELISA

After three rounds of panning, ARI 292 cells were infected with the eluted phage and grown on LB-Agar Ampicillin plates. Ten individual clones were picked and used for inoculation of tubes with 5 ml of SOP with ampicillin and glucose as described above; cells were grown with shaking at 37° C. After cultures reached an OD₆₀₀=0.5 AU, 1 μl of M13K07 helper phage at a concentration of 1×10¹¹ transducing units/ml was added for growing individual recombinant phage supernatants. Cultures were then incubated for 30 minutes at 37° C. without shaking to allow infection of the cells. Arabinose and kanamycin were added as described above, and cultures were grown overnight with shaking at 37° C. The next day, the cells were pelleted by centrifugation at 10,000 rpm for 5 minutes and the supernatants were used for screening. ELISA screening for phage that reacted with the PBA-antibody complex was performed by direct addition of 50 μl of supernatants to wells coated with 0.5 μg/well of affinity purified anti-PBA polyclonal antibody, with or without addition of 50 μl of 200 ng/ml of phenoxybenzoic acid per well.

### Stabilization of Phage Suspensions

Individual amplified phage clones were obtained as described above. After two steps of precipitation with PEG-NaCl, the phage particles were suspended in 1/50 volume of the original culture volume in PBS, which was supplemented with Complete Protease Inhibitor Cocktail (Roche Diagnostics) and 0.05% sodium azide. The preparation was filtered through a 0.22 μm filter and stored in aliquots at 4° C. and −80° C.

### Noncompetitive Phage ELISA

ELISA plates were coated with the gamma-globulin fraction of the anti-PBA rabbit serum purified on Protein G columns (Amersham-Pharmacia, Uppsala) using 100 μl of 10, 5, 2.5 and 1.25 μg/ml in PBS. After incubation for 1 hour at 37° C. and blocking for 1 hour at 37° C. with 1% BSA, the plates were washed 3 times with PBS-0.05% Tween 20. Serial 2-fold dilutions of phage preparation (starting from a 1/50 dilution) were performed in PBS-0.05% Tween 20 in non-treated polystyrene plates (low binding capacity) in the presence (50 ng/ml) of PBA or in its absence. The phage dilutions were then transferred to wells pre-coated with the protein G-purified antibody. After incubation for 1 h at 37° C., the plates were washed and 100 μl of the appropriate dilution of an anti-rabbit IgG antibody coupled to peroxidase was added to the wells, incubated for 30 min at 37° C. and thoroughly washed. The peroxidase activity was then developed by adding 100 μl of peroxidase substrate (25 ml of 0.1 M citrate acetate buffer pH 5.5, 0.4 ml of 6 mg/ml DMSO solution of 3,3′,5,5′-tetramethylbenzidine and 0.1 ml of 1% H₂O₂), which was dispensed into each well. The enzymatic reaction was stopped after 15-20 min by the addition of 50 μl of 2M H₂SO₄, and the absorbance at 450 nm (corrected at 600 nm) was read in a microtiter plate reader (Multiskan MS, Labsystems). Absorbance values were fitted to a four-parameter logistic equation using Genesis Lite 3.03 (Life Sciences (UK) Ltd.) package software.

### Cross-Reactivity Assay

The specificity of the noncompetitive assay set up with the phage borne peptide was characterized by determining its cross-reactivity with structurally related pesticides. PBA concentrations in the 0-50 ng/ml range were used in the noncompetitive ELISAs. Data were normalized, and the molar compound concentration corresponding to the midpoint of the curve, (which corresponds to the concentration of analyte producing 50% saturation of the signal (SC₅₀), was used to express the cross-reactivity of the assay according to the equation:

% cross−reactivity=100×[SC₅₀(PBA)/SC₅₀(cross−reacting compound)]

### Dipstick Assay

One and a half μl of serial dilutions of protein G-purified anti-PBA antibodies in PBS were spotted onto a 0.45-micron nitrocellulose membrane (Biorad Laboratories Inc., CA). Immediately after drying, the membrane was quenched by soaking into 5% skimmed milk in PBS for 30 min; after the membrane was rinsed in PBST, 5 mm strips were cut, dried and kept at 4° C. until used. The assay was performed by dipping the membrane strips for 15 min into water spiked with different amounts of PBA and containing the appropriate dilution of phage, washed under tap water, incubated for 10 min in a diluted solution of anti-M13 peroxidase conjugate, washed under tap water, and developed using the diaminobenzidine-nickel chloride substrate mix.

### B. Results and Discussion

Panning a Peptide Library with Polyclonal Antibodies Against PBA

A phage display peptide library expressing random octapeptides flanked by a disulfide bridge and fused to the phage protein pIII was panned as described above. In order to favor the isolation of peptides that recognize the PBA-antibody IC, an excess of PBA was included in the panning experiments. After 3 rounds of panning, 10 individual clones were tested for binding to the affinity purified anti-PBA antibody coated wells in the presence or absence of PBA as described in Materials and Methods. As shown in FIG. 9, 9 out of 10 clones exhibited stronger binding to the polyclonal antibodies in the presence of PBA (100 ng/ml) than in its absence. Sequencing of the 9 reactive clones revealed that all of them coded for the same peptide sequence, CFNGKDWLYC.

### Noncompetitive ELISA

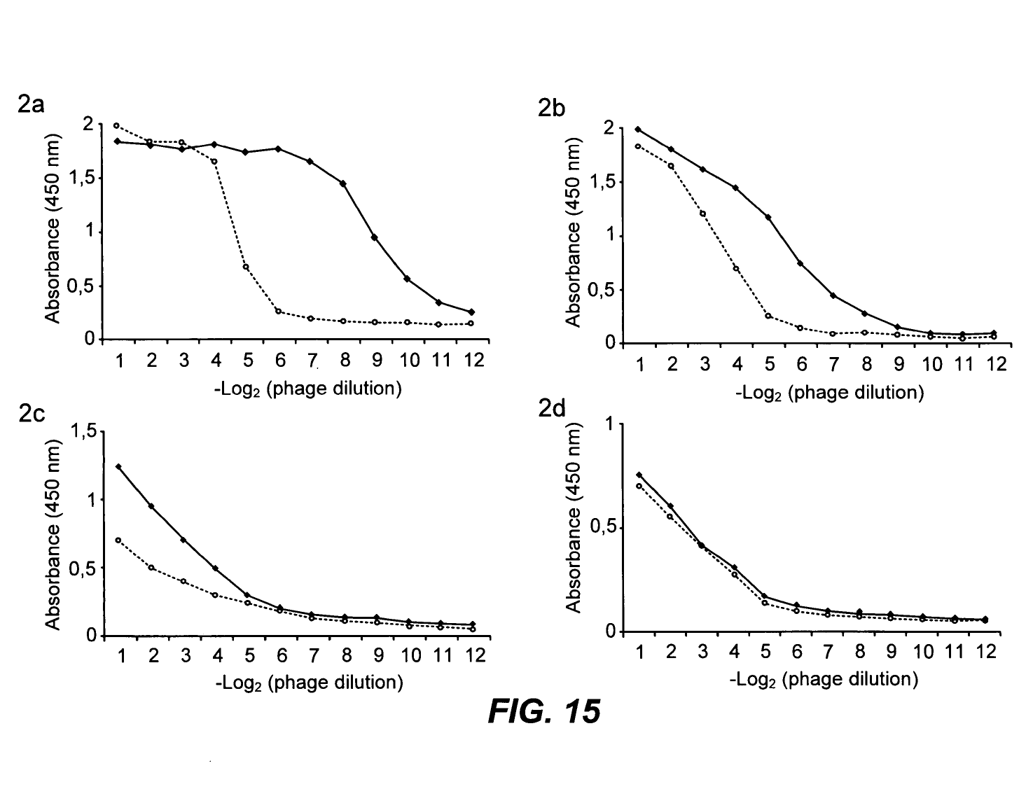

One of the phage clones displaying the CFNGKDWLYC sequence was amplified and prepared as a stabilized phage suspension as described above (8×10¹² transducing units/ml). This phage solution was used to determine the antibody-coating conditions that allowed maximization of the differential signal in the presence or absence of PBA. As shown in FIG. 15, both the amount of polyclonal antibody used for coating and the concentrations of phage particles exerted an influence on differential binding to the IC. An optimum was obtained when 100 μl of 10 μg/ml protein G-purified anti-PBA antibody was used for coating, in combination with 2.5×10⁹ phage transducing units/ml. These conditions were used to set up the noncompetitive PHAIA shown in FIG. 11. The dose-response binding curve had a typical sigmoidal shape with signal saturation at high concentrations of analyte. The detection limit of the assay, estimated from the reading of zero analyte concentration plus four standard deviations, was 0.05 ng/ml, and the working range was 0.05-3.0 ng/ml. The midpoint of the curve, corresponding to the concentration of analyte at which ELISA readings are 50% of the maximal signal (SC₅₀), was SC₅₀=0.31±0.03 ng/ml. This represents a 5-fold improvement in assay sensitivity as compared to the midpoint of the heterologous competitive ELISA set up with the same polyclonal antibodies reported by Shan et al., which exhibited a 50% inhibition concentration of 1.65 ng/ml (see, e.g., Shan, G.; Huang, H.; Stoutamire, D. W.; Gee, S. J.; Leng, G.; Hammock, B. D. Chem. Res. Toxicol., 2004, 17, 218-225).

### PBA PHAIA Cross-Reactivity

The cross-reactivity of the PHAIA for PBA was assessed using PBA related compounds, including several common pyrethroids, as shown in Table 6. In all cases, the cross-reactivity was negligible.

TABLE 6
RESULTS OF CROSS-REACTIVITIES OF NONCOMPETITIVE PHAGE ELISA FOR PBA

| Chemicals | Structures | IC₅₀ (ng/mL) | CR (%) |
|---|---|---|---|
| PBA | 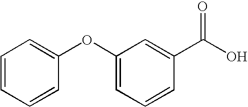 | 0.3 | 100 |
| PBA-glycine conjugate | 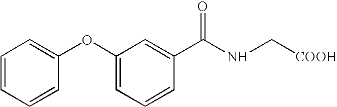 | >100 | 0 |
| 4-Hydroxybenzoic acid | 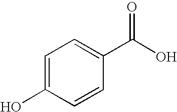 | >100 | 0 |
| Permethrin | 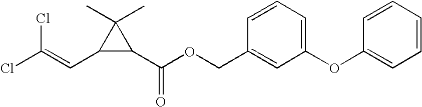 | >100 | 0 |
| Cypermethrin | 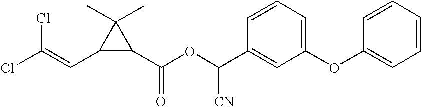 | >100 | 0 |
| Esfenvalerate | 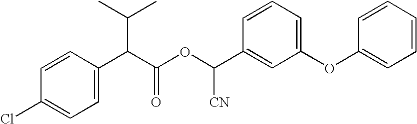 | >100 | 0 |

### Matrix Effect

PBA is a common metabolite of many major pyrethroid pesticides and its presence in urine is a valuable biomarker of human exposure to these compounds (see, e.g., Leng, G.; Leng, A.; Kuhn, K. H.; Lewalter, J.; Pauluhn, J. Xenobiotica, 1997, 27, 1273-1283; Saieva, C.; Aprea, C.; Tumino, R.; Masala, G.; Salvini, S.; Frasca, G.; Giurdanella, M. C.; Zanna, I.; Decarli, A.; Sciarra, G.; Palli, D. Sci Total Environ, 2004, 332, 71-80). For this reason we examined whether there was a urine matrix effect upon the performance of the PBA PHAIA. Whether there was an effect was evaluated by monitoring the recovery of spiked PBA in urine measured by the noncompetitive phage ELISA (Table 7). These experiments showed a good correlation and almost no matrix effect when up to a 10% final concentration of urine was used.

TABLE 7
Relation between spiked PBA in
urine and measure by noncompetitive
PHAIA

| Final | | | |
|---|---|---|---|
| concentration | Spiked PBA | | Mean Recovery |
| of urine (%) | (ng/ml) | Measured PBA (ng/ml) | (%, n = 3) |
| 0 | 1 | 1.1 ± 0.1 | 115 |
| | 2 | 1.6 ± 0.4 | 82 |
| 1 | 1 | 1.2 ± 0.2 | 124 |
| | 2 | 2.3 ± 0.1 | 115 |
| 2.5 | 1 | 1.2 ± 0.2 | 118 |
| | 2 | 2.0 ± 0.2 | 98 |
| 5 | 1 | 1.1 ± 0.2 | 106 |
| | 2 | 2.2 ± 0.1 | 108 |
| 10 | 1 | 1.1 ± 0.2 | 109 |
| | 2 | 1.7 ± 0.2 | 85 |

Adaptation of PHAIA into a Dipstick Format

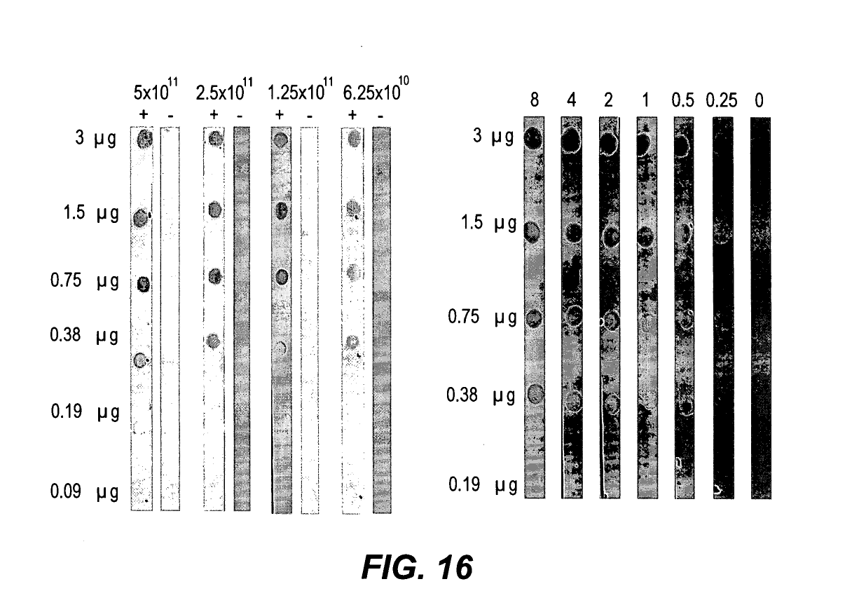

One of the contexts in which direct readings are preferred over indirect readings is when the assay needs to be adapted into an ‘on site’ rapid test such as dipsticks or immunochromatography. In these formats, the assay sensitivity is favored by the fact that the visual reading produced by trace amounts of the analyte can be easily discriminated from the zero signal. For this reason, we adapted the PHAIA into a dipstick format using protein G-isolated antibodies immobilized onto nitrocellulose strips, as described above. As shown in the left panel of FIG. 16, there was no background reactivity in a wide range of antibody coating densities for different phage concentrations. Similarly, there was not detectable reactivity of the phage borne peptide with the unliganded antibody with all concentrations of phage examined. Thus, working dilutions could be chosen on the basis of the desired sensitivity, economy of the reagents and intensity of the signal. In this case, we used 5×10¹¹ phage particles/ml for detection. The sensitivity of the assay in a dipstick format is shown in the right panel of FIG. 16. Detection of as low as 0.25 ng/ml was obtained with most of the antibody coating conditions tested, with the clearest signal at high antibody coating density (3 μg/dot).

### C. Conclusion

A general technique is described here for specific and sensitive noncompetitive detection of small size analytes based on the use of phage borne peptides isolated from phage libraries and polyclonal antibodies. This method is an advantageous alternative to the preparation of anti-metatype antibodies for several reasons, among which are: 1) Anti-metatype antibodies are difficult to prepare, which is reflected by the limited number of applications that have been reported after the principle was first described (see, e.g., Ullman, E. F.; Milburn, G.; Jelesko, J.; Radika, K.; Pirio, M.; Kempe, T.; Skold, C. Proc Natl Acad Sci USA, 1993, 90, 1184-1189; Self, C. H.; Dessi, J. L.; Winger, L. A. Clin Chem, 1994, 40, 2035-2041). The main drawback to the anti-metatype antibody approach appears to be the strong cross-reactivity with the empty binding site of the capture antibody, which makes use of anti-metatype antibodies for noncompetitive ELISA an arduous trial and error process (see, e.g., Ullman, E. F.; Milburn, G.; Jelesko, J.; Radika, K.; Pirio, M.; Kempe, T.; Skold, C. Proc Natl Acad Sci USA, 1993, 90, 1184-1189). PHAIA technology avoids the immunization protocols and monoclonal antibody preparation that are necessary to obtain these anti-metatype antibodies, substituting for them a rapid selection process from phage display peptide libraries, which can be prepared in house or bought from commercial suppliers; 2) One strength of the present invention is its general applicability. Phage display peptide libraries offer a huge chemical diversity as starting point, which surpasses by many orders of magnitude the number of hybridoma clones that could be practically tested in the search for anti-metatype antibodies. In addition, the selected short peptide loops present a smaller binding surface than anti-IC antibodies, which allows focusing the recognition of the IC to the changes produced upon binding of the analyte. Therefore, a higher control over the background cross-reactivity with the unliganded antibody is a feature of this approach; 3) The use of trivalent detection of an analyte increases assay sensitivity, as observed in this study, because the ternary complex has a higher overall affinity, as has been observed for anti-metatype/IC complexes (see, e.g., Ullman, E. F.; Milburn, G.; Jelesko, J.; Radika, K.; Pirio, M.; Kempe, T.; Skold, C. Proc. Natl. Acad. Sci. USA, 1993, 90, 1184-1189); 4) The present invention can be applied when the capture antibody is polyclonal in nature. Polyclonal antibodies are easy to prepare and are often the first choice in the development of immunoassays for small analytes. However, due to their heterogeneity, they can not be used as immunizing IC for the preparation of anti-metatype antibodies. PHAIA technology w removes this limitation and allows the development of polyclonal antibody-based noncompetitive immunoassays. While the examples shown here employed affinity purification of the polyclonal antibodies for the panning experiments, this can be easily accomplished by immobilization of the immunizing hapten used to raise the antibodies; 5) Also, it has been shown that in general, competitive polyclonal based immunoassays attain maximum sensitivity when the immunizing and competing haptens are different (heterologous assays). This requires extensive chemical synthesis work in order to develop a proper panel of candidate haptens, which must afterwards be tested to examine whether the desired sensitivity can be reached. This inefficient trial and error process can be completely avoided by application of the PHAIA, which thus, not only allows a positive reading, but also provides a convenient shortcut in the development of polyclonal based immunoassays.

The foregoing examples demonstrate that the methods described here can be used as a general approach for the development of noncompetitive assays to detect analytes. In particular, although the invention has been exemplified above using three particular examples of small molecules, it would be appreciated by the skilled artisan that the methods of the present invention are generally applicable to any analyte for which for which an antibody, monoclonal or polyclonal, can be generated. The methods of the present invention are also broadly applicable to other instances of molecular binding, such as ligand-receptor binding or other protein-protein interactions.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. All publications, patents, and patent applications cited herein are hereby incorporated by reference in their entirety for all purposes.

What is claimed is:

1. A method for detecting an analyte, said method comprising:
(a) contacting a sample suspected of containing said analyte with a first antibody that specifically binds to the analyte, thereby forming an antibody-analyte complex;
(b) contacting the antibody-analyte complex with an affinity agent comprising a phage protein fused to a heterologous polypeptide, wherein the heterologous polypeptide specifically binds to the antibody-analyte complex,
thereby forming an antibody-analyte-affinity agent complex; and
(c) detecting the antibody-analyte-affinity agent complex, thereby detecting the analyte.

2. The method of claim 1, wherein the analyte is a small molecule.

3. The method of claim 1, wherein the analyte is an epitope for an antibody.

4. The method of claim 1, wherein said first antibody is a monoclonal antibody.

5. The method of claim 1, wherein said affinity agent comprises phage coat protein pVIII.

6. The method of claim 1, wherein said affinity agent comprises phage coat protein pIII.

7. The method of claim 5 or 6, wherein the antibody-analyte-affinity agent complex is detected by contacting the complex of step (b) with a second antibody that specifically binds to a phage protein.

8. The method of claim 7, wherein said second antibody further comprises a detectable label.

9. The method of claim 8, wherein said detectable label is a fluorescent label.

10. The method of claim 1, wherein said analyte is selected from the group consisting of: molinate, atrazine, and phenoxybenzoic acid.

11. The method of claim 10, wherein said analyte is molinate and said heterologous polypeptide comprises the following sequence: WDT.

12. The method of claim 11, wherein said heterologous polypeptide comprises the following sequence: X₁X₂X₃WDTX₄X₅, wherein X₁ is selected from the group consisting of: C and R; X₂ is selected from the group consisting of: S and N; X₃ is selected from the group consisting of: T, R, H, and V; X₄ is selected from the group consisting of: T, W, and S; and X₅ is selected from the group consisting of: G and C.

13. The method of claim 10, wherein said analyte is atrazine and said polypeptide comprises the following sequence: WFD.

14. The method of claim 13, wherein said heterologous polypeptide comprises the following sequence: X₁WFDX₂X₃, wherein X₁ is selected from the group consisting of: R, S, Y, and M; X₂ is selected from the group consisting of: N, E, A, L, and M; and X₃ is selected from the group consisting of: S, G, P, C, and Y.

15. The method of claim 13, wherein said heterologous polypeptide comprises the following sequence: X₁WFDX₂X₃, wherein X₁ is selected from the group consisting of: R, S, Y, and M; X₂ is selected from the group consisting of: N, E, A, L, and M; and X₃ is selected from the group consisting of: S, G, P, C, and Y.

16. The method of claim 10, wherein said analyte is phenoxybenzoic acid and said polypeptide comprises the following sequence: CFNGKDWLYC.

17. A kit for detecting an analyte, said kit comprising:
(a) a first antibody that specifically binds to the analyte; and
(b) an affinity agent comprising a phage protein fused to a heterologous polypeptide, wherein the heterologous polypeptide specifically binds to a complex between the first antibody and the analyte.

18. The kit of claim 17, wherein the first antibody is a monoclonal antibody.

19. The kit of claim 17, wherein said analyte is selected from the group consisting of: molinate, atrazine, and phenoxybenzoic acid.

20. The kit of claim 17, further comprising a second antibody that specifically binds to a phage protein.

21. The kit of claim 17, wherein said first antibody is immobilized to a solid support.

22. The kit of claim 21, wherein said solid support is selected from the group consisting of a membrane and multiwell plate.

23. The kit of claim 22, wherein the membrane is nitrocellulose.

24. The kit of claim 21, wherein the solid support is a dipstick.
